(19) United States
(12) Patent Application Publication
(10) Pub. No.: US 2004/0137455 A1
(43) Pub. Date: Jul. 15, 2004

(54) Diagnosis of cancer or benign tumor using the aberrant expression product of the klk4 gene

(76) Inventors: Ying Dong, Queensland (AU); Judith Ann Clements, Queensland (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/473,485

(22) PCT Filed: Mar. 27, 2002
(86) PCT No.: PCT/AU02/00378

(30) Foreign Application Priority Data
Mar. 27, 2001 (AU) PR 4022

(51) Int. Cl. C12Q 1/68; G01N 33/574

(52) U.S. Cl. 435/6; 435/7.23

(57) ABSTRACT

The present invention discloses aberrant expression products of the KLK4 gene, which segregate with at least one condition selected from a cancer or a benign tumour. The invention also discloses a method for detecting the presence or diagnosing the risk of said at least one condition by detecting aberrant KLK4 expression The invention also discloses isolated polynucleotides comprising a nucleotide sequence that corresponds or is complementary to at least a portion of an aberrant KLK4 polynucleotide, which correlates with the presence or risk of said at least one condition. Also disclosed are isolated polypeptides comprising an amino acid sequence that corresponds to at least a portion of an aberrant K4 polypeptide, which correlates with the presence or risk of said at least one condition. The invention also extends to variants and derivatives of these molecules, to vectors comprising aberrant KLK4 polynucleotides and to host cells containing such vectors. The invention further extends to antigen-binding molecules that are immuno-interactive with aberrant K4 polypeptides and to the use of these antigen-binding molecules, the aberrant KLK4 polynucleotides and aberrant K4 polypeptides in assays and kits for detecting the presence or diagnosing the risk of said at least one condition. The invention further encompasses the use of functional KLK4 polynucleotides or functional K4 polypeptides or agents that modulate the level and/or functional activity of an expression product of KLK4 or of a gene belonging to the same biosynthetic or regulatory pathway as KLK4 for treating and/or preventing one or more of said conditions.

### FIELD OF THE INVENTION

THIS INVENTION relates generally to polynucleotides and polypeptides linked to cancer and/or benign tumours. More particularly, the present invention relates to aberrant expression products of the KLK4 gene, which segregate with at least one condition selected from a cancer or a benign tumour. Even more particularly, the present invention relates to isolated polynucleotides comprising a nucleotide sequence that corresponds or is complementary to at least a portion of an aberrant KLK4 polynucleotide that correlates with the presence or risk of said at least one condition as well as to isolated polypeptides that correspond to at least a portion of an aberrant K4 polypeptide that correlates with the presence or risk of said at least one condition. The invention also extends to variants and derivatives of these molecules, to vectors comprising aberrant KLK4 polynucleotides and to host cells containing such vectors. The invention further extends to antigen-binding molecules that are immuno-interactive with aberrant K4 polypeptides and to the use of these antigen-binding molecules, the aberrant KLK4 polynucleotides and aberrant K4 polypeptides in assays and kits for detecting the presence or diagnosing the risk of said at least one condition. The present invention also relates to a method for detecting the presence or diagnosing the risk of said at least one condition, either before or after the onset of clinical symptoms, by detecting aberrant KLK4 expression. The invention further encompasses the use of functional KLK4 polynucleotides or functional K4 polypeptides or agents that modulate the level and/or functional activity of an expression product of KLK4 or of a gene belonging to the same biosynthetic or regulatory pathway as KLK4 for treating and/or preventing one or more of said conditions.

Bibliographic details of the publications numerically referred to in this specification are collected at the end of the description.

### BACKGROUND OF THE INVENTION

The KLKs are a highly conserved gene family for serine proteases involved in a number of physiological and pathophysiological events, such as the regulation of local blood flow, angiogenesis, cell proliferation, extracellular matrix (ECM) degradation and mitogenesis (1, 2). This gene family has also be shown to be involved in tumorigenesis, primarily of the prostate and breast, and to be regulated by oestrogen and progesterone, as well as androgens, in a number of systems (1-3).

Previous studies have shown that KLK1-3 are expressed in the human endometrium (4) and implicated in various aspects of uterine function (1, 4, 5) whilst KLK1 is also expressed in endometrial cancer tissue (6). Other more recent studies have shown that KLK6, KLK7 and KLK8 (7-9) (formerly known as protease M, stratum corneum chymotryptic enzyme and neuropsin, respectively) are highly expressed in ovarian carcinomas and that KLK4 (10-13) (formerly known as prostase, KLK-L1, PRSS17) is expressed in the prostate cancer cell line LNCaP (10) and the breast cancer cell line BT-474 (11). Moreover, the expression of KLK4 mRNA (10, 11) has been shown to be up-regulated by androgens, progestins, or oestrogen in these cell lines.

### SUMMARY OF THE INVENTION

The present invention is predicated in part on the discovery that sequence alterations within the coding regions of KLK4, inclusive of substitutions, deletions and additions, are linked to ovarian and endometrial cancer. It has also been surprisingly discovered that K4, the polypeptide encoded by KLK4, and/or aberrant K4 polypeptides, are highly expressed in ovarian and endometrial cancers relative to normal ovarian and endometrial tissues. Based on pathophysiologies associated with other members of the KLK gene family, the present inventors believe that aberrant expression of KLK4 may potentially relate to other hormone-associated carcinomas, including breast cancer and prostate cancer. The foregoing discoveries have been reduced to practice in methods of diagnosing various conditions associated with aberrant expression of KLK4, in new isolated molecules for use in such diagnosis, and in compositions for treating and/or preventing the aforesaid conditions as described hereinafter.

Accordingly, in one aspect of the present invention, there is provided a method for detecting the presence or diagnosing the risk of at least one condition selected from a cancer or a benign tumour in a patient, comprising detecting aberrant expression of KLK4 in a biological sample obtained from said patient. The cancer or benign tumour is preferably associated with an organ or tissue selected from ovaries, endometrium or prostate. The condition is suitably regulatable by a hormone including, but not restricted to, testosterone, oestrogen and progesterone. Preferably, the condition is a cancer selected from ovarian, endometrial or prostate cancer.

Aberrant expression of KLK4 is preferably detected by detecting a change in the level and/or functional activity of an expression product of a gene selected from KLK4 or a gene belonging to the same regulatory or biosynthetic pathway as KLK4, wherein the change is relative to a normal reference level and/or functional activity. In one embodiment of this type, the change in the level and/or functional activity of said expression product is detected in a basal cell, which is preferably of prostatic origin. In another embodiment of this type, the change in the level and/or functional activity of said expression product is detected in a stem cell, which is preferably of prostatic origin and is a precursor of, or differentiates into, an epithelial cell or a malignant cancer cell. In yet another embodiment of this type, the change in the level and/or functional activity of said expression product is detected in a precursor lesion to a cancer. In another embodiment, the change in the level and/or functional activity of said expression product is detected in a prostatic intra-epithelial neoplasia (PIN). In still yet another embodiment of this type, the change in the level and/or functional activity of said expression product is detected in a bone metastasis, which is preferably associated with an ovarian cancer or an endometrial cancer and more preferably with a prostate cancer. In another embodiment of this type, the change in the level and/or functional activity of said expression product is detected in the nucleus of a cell, which is preferably an endometrial cell and more preferably a prostate cell or an ovarian cell.

In a related aspect, the present invention provides a method for detecting the presence or diagnosing the risk of at least one condition selected from a cancer or a benign tumour in a patient, comprising determining the presence of an aberrant KLK4 expression product in a biological sample obtained from said patient, wherein said aberrant expression product correlates with the presence or risk of said at least one condition. The aberrant expression product is suitably selected from an aberrant K4 polypeptide with impaired, altered or abrogated function relative to normal K4, or an aberrant K4 polynucleotide encoding said aberrant K4 polypeptide.

The aberrant K4 polypeptide may comprise a substitution, deletion and/or addition of one or more amino acids relative to normal K4 and preferably comprises the sequence set forth in any one of SEQ ID NO: 2, 4 and 15. Suitably, the presence of an aberrant K4 polypeptide is detected in the nucleus of a cell, which is preferably an endometrial cell and more preferably a prostate cell or an ovarian cell. In one embodiment of this type, the aberrant K4 polypeptide has a molecular weight that is lower than the molecular weight of a K4 polypeptide present in the nucleus of a normal cell. In another embodiment of this type, the aberrant K4 polypeptide comprises an insertion relative to normal K4, which insertion preferably comprises the sequence set forth in SEQ ID NO: 9. In yet another embodiment of this type, the aberrant K4 polypeptide comprises the sequence set forth in SEQ ID NO: 2.

The aberrant KLK4 polynucleotide may comprise a substitution, deletion and/or addition of one or more nucleotides relative to normal KLK4 and preferably comprises the sequence set forth in any one of SEQ ID NO: 1, 3 and 14. In one embodiment of this type, the presence of said aberrant KLK4 polynucleotide or said expression product is detected in a basal cell, which is preferably of prostatic origin. In another embodiment of this type, the presence of said aberrant KLK4 polynucleotide or said expression product is detected in a stem cell of prostatic origin which is suitably a precursor of, or differentiates into, an epithelial cell or a malignant cancer cell. In yet another embodiment of this type, the presence of said aberrant KLK4 polynucleotide or said expression product is detected in a precursor lesion to cancer. In still yet another embodiment of this type, the presence of said aberrant KLK4 polynucleotide or said expression product is detected in a prostatic intra-epithelial neoplasia (PIN). In a further embodiment of this type, the presence of said aberrant KLK4 polynucleotide or said expression product is detected in a bone metastasis.

In another aspect, the invention encompasses the use of an aberrant KLK4 polynucleotide encoding an aberrant K4 polypeptide with impaired, altered or abrogated function relative to normal K4 or the use of said aberrant K4 polypeptide or the use of an antigen-binding molecule that is immuno-interactive specifically with said aberrant K4 polypeptide in the manufacture of a kit for detecting an aberrant KLK4 polynucleotide or an aberrant K4 polypeptide, which correlate with the presence or risk of a condition selected from a cancer or a benign tumour.

In yet another aspect, the invention provides a method for restoring K4 function in a patient whose level and/or functional activity of normal or wild-type K4 is reduced or abrogated, comprising administering to said patient an effective amount of a functional KLK4 polynucleotide or a biologically active fragment thereof, or a functional K4 polypeptide or a biologically active fragment thereof.

In still another aspect, the invention provides a method of treating or preventing the development of a condition selected from a cancer or a benign tumour, comprising administering to a patient in need of such treatment an effective amount of a functional KLK4 polynucleotide or a biologically active fragment thereof, or a functional K4 polypeptide or a biologically active fragment thereof.

In yet another aspect, the invention contemplates the use of an agent in the manufacture of a medicament for restoring a normal level and/or functional activity of a KLK4 expression product in a patient having an aberrant or abnormal level and/or functional activity of said expression product, wherein said agent is optionally formulated with a pharmaceutically acceptable carrier and modulates the expression of a gene or the level and/or functional activity of an expression product of said gene, wherein said gene is selected from KLK4 or a gene belonging to the same regulatory or biosynthetic pathway as KLK4, and is identifiable by a screening assay comprising:

contacting a preparation comprising a polypeptide encoded by said gene, or a biologically active fragment of said polypeptide, or a variant or derivative of these, or a genetic sequence that modulates the expression of said gene, with said agent; and

detecting a change in the level and/or functional activity of said polypeptide or biologically active fragment, or variant or derivative, or of a product expressed from said genetic sequence.

Preferably, the patient has an elevated level of said expression product and said agent reduces the level and/or functional activity of said polypeptide or biologically active fragment thereof, or variant or derivative, or of said expression product. In a preferred embodiment of this type, the agent is an antigen-binding molecule that is immuno-interactive with a K4 polypeptide.

In still yet another aspect, the invention contemplates the use of an agent in the manufacture of a medicament for modulating the level and or functional activity of an aberrant KLK4 expression product in a patient, wherein said agent is optionally formulated with a pharmaceutically acceptable carrier and is identifiable by a screening assay comprising:

contacting a preparation comprising an aberrant K4 polypeptide, or a biologically active fragment thereof, or a variant or derivative of these, or an aberrant KLK4 transcript, with said agent; and

detecting a change in the level and/or functional activity of said polypeptide or biologically active fragment, or variant or derivative, or said aberrant KLK4 transcript.

In one embodiment of this type, the agent is an antigen-binding molecule that is immuno-interactive with an aberrant K4 polypeptide. In another embodiment of this type, the agent is an antisense oligonucleotide or ribozyme that binds to, or otherwise interacts specifically with, an aberrant KLK4 transcript.

In still yet another aspect, the invention contemplates the use of an agent in the manufacture of a medicament for treating and/or preventing at least one condition selected from a cancer or a benign tumour, wherein said agent is optionally formulated with a pharmaceutically acceptable carrier and modulates the expression of a gene or the level and/or functional activity of an expression product of said gene, wherein said gene is selected from KLK4 or a gene belonging to the same regulatory or biosynthetic pathway as KLK4, and is identifiable by a screening assay comprising:

contacting a preparation comprising a polypeptide encoded by said gene, or biologically active fragment of said polypeptide, or variant or derivative of these, or a genetic sequence that modulates the expression of said gene, with said agent; and

detecting a change in the level and/or functional activity of said polypeptide or biologically active fragment thereof, or variant or derivative, or of a product expressed from said genetic sequence.

In still yet another aspect, the invention features a method for restoring a normal level of a KLK4 expression product in a patient in need of such treatment, comprising administering to said patient an effective amount of an agent as broadly described above and optionally a pharmaceutically acceptable carrier.

According to another aspect, the invention provides a method for the treatment and/or prophylaxis of at least one condition selected from a cancer or a benign tumour, comprising administering to a patient in need of such treatment an effective amount of an agent as broadly described above and optionally a pharmaceutically acceptable carrier.

In another aspect, the invention contemplates an isolated polynucleotide comprising a nucleotide sequence which corresponds or is complementary to at least a portion of an aberrant KLK4 polynucleotide that correlates with the presence or risk of at least one condition selected from a cancer or a benign tumour.

The aberrant KLK4 polynucleotide preferably encodes an aberrant K4 polypeptide having altered, impaired or abrogated function relative to a normal K4 polypeptide. For example, the aberrant KLK4 polynucleotide may comprise a substitution, deletion and/or addition of one or more nucleotides in an open reading frame of a normal KLK4 polynucleotide. Preferably, the aberrant KLK4 polynucleotide is an alternately spliced variant of normal KLK4.

The at least a portion of said aberrant KLK4 polynucleotide suitably comprises at least 10, preferably at least 15, more preferably at least 18 and even more preferably at least 20 nucleotides.

In one embodiment, the aberrant KLK4 polynucleotide comprises all or part of the intron located between exon 3 and exon 4 of normal KLK4 as set forth in SEQ ID NO: 12. Preferably, the aberrant KLK4 polynucleotide comprises the nucleotide sequence set forth in SEQ ID NO: 7. In this instance, the aberrant KLK4 polynucleotide preferably comprises a 3′ coding sequence comprising the sequence set forth in SEQ ID NO: 8. In a preferred embodiment of this type, the aberrant KLK4 polynucleotide comprises the sequence set forth in SEQ ID NO: 1.

In another embodiment, the aberrant KLK4 polynucleotide comprises a deletion corresponding to all or part of exon 4 of normal KLK4 as set forth in SEQ ID NO: 12. Preferably, the deletion comprises all or part of the sequence set forth in SEQ ID NO: 10. Suitably, the aberrant KLK4 polynucleotide comprises all or part of a sequence corresponding to exon 3 of normal KLK4 spliced together with all or part of a sequence corresponding to exon 5 of normal KLK4. In a preferred embodiment of this type, the aberrant KLK4 polynucleotide comprises the sequence set forth in SEQ ID NO: 18. In an especially preferred embodiment, the aberrant KLK4 polynucleotide comprises the sequence set forth in SEQ ID NO: 3.

In yet another embodiment, the aberrant KLK4 polynucleotide comprises all or part of the intron located between exon 2 and exon 3 of normal KLK4 as set forth in SEQ ID NO: 17. In a preferred embodiment of this type, the aberrant KLK4 polynucleotide comprises the intronic sequence set forth in SEQ ID NO: 16. Suitably, the aberrant KLK4 polynucleotide further comprises a deletion corresponding to all or part of exon 4 of normal KLK4 as set forth in SEQ ID NO: 12. Suitably, the aberrant KLK4 polynucleotide comprises all or part of a sequence corresponding to exon 3 of normal KLK4 spliced together with all or part of a sequence corresponding to exon 5 of normal KLK4. In a preferred embodiment of this type, the aberrant KLK4 polynucleotide comprises the sequence set forth in SEQ ID NO: 18. In an especially preferred embodiment, the aberrant KLK4 polynucleotide preferably comprises the nucleotide sequence set forth in SEQ ID NO: 14.

Preferably, the aberrant KLK4 polynucleotide is selected from the group consisting of:

(a) a polynucleotide comprising the entire sequence of nucleotides set forth in SEQ ID NO: 1;

(b) a polynucleotide fragment of (a), wherein said fragment comprises SEQ ID NO: 7 or fragment thereof;

(c) a polynucleotide comprising the entire sequence of nucleotides set forth in SEQ ID NO: 3;

(d) a polynucleotide fragment of (c), wherein said fragment comprises the codon spanning nucleotides 475 through 477 of SEQ ID NO: 3;

(e) a polynucleotide fragment of (c), wherein said fragment comprises all or part of a sequence corresponding to exon 3 of normal KLK4 spliced together with all or part of a sequence corresponding to exon 5 of normal KLK4;

(f) a polynucleotide comprising the entire sequence of nucleotides set forth in SEQ ID NO: 14;

(g) a polynucleotide fragment of (f), wherein said fragment-comprises SEQ ID NO: 17, or portion thereof;

(h) a polynucleotide fragment of (f), wherein said fragment comprises the codon spanning nucleotides 223 through 225 of SEQ ID NO: 14; and

(i) a polynucleotide fragment of (f), wherein said fragment comprises all or part of a sequence corresponding to exon 3 of normal KLK4 spliced together with all or part of a sequence corresponding to exon 5 of normal KLK4.

The present invention also encompasses a method of identifying aberrant expression products, which correlate with the presence or risk of at least one condition selected from a cancer or a benign tumour, comprising determining the sequence of a KLK4 expression product from subjects known to have said at least one condition and comparing the sequence to that of wild-type KLK4 expression products to thereby identify said aberrant expression products.

The invention in yet another aspect contemplates a probe for interrogating nucleic acid for the presence of an aberrant KLK4 polynucleotide associated with at least one condition selected from a cancer or a benign tumour, comprising a nucleotide sequence which corresponds or is complementary to a portion of an aberrant KLK4 polynucleotide that correlates with the presence or risk of said at least one condition.

In yet another aspect, the invention provides a vector comprising an isolated polynucleotide as broadly described above, or a probe as broadly described above.

In still another aspect, the invention encompasses an expression vector comprising an isolated polynucleotide as broadly described above, operably linked to a regulatory polynucleotide.

In another aspect, the invention provides a host cell containing a vector or expression vector as broadly described above.

In yet another aspect, the invention provides a cell line comprising an aberrant KLK4 polynucleotide as broadly described above. Preferably, the cell line is derived from a patient who has a condition selected from a cancer or a benign tumour.

In another aspect, the invention encompasses an isolated polypeptide comprising an amino acid sequence which corresponds to at least a portion of an aberrant K4 polypeptide that correlates with the presence or risk of at least one condition selected from a cancer or a benign tumour.

The aberrant polypeptide suitably has altered, impaired or abrogated function relative to a normal K4. For example, the aberrant K4 polypeptide may comprise a substitution, deletion and/or addition of one or more amino acids relative to normal K4.

In one embodiment, the aberrant K4 polypeptide comprises an insertion relative to normal K4, which preferably comprises the sequence set forth in SEQ ID NO: 9. More preferably, the aberrant K4 polypeptide comprises the sequence set forth in SEQ ID NO: 2.

In another embodiment, the aberrant K4 polypeptide comprises a truncation relative to normal K4, wherein said truncation is associated with a deletion of all or part of the amino acid sequence set forth in SEQ ID NO: 11. More preferably, the aberrant K4 polypeptide comprises the sequence set forth in SEQ ID NO: 4.

In yet another embodiment, the aberrant K4 polypeptide comprises a truncation relative to normal K4, wherein said truncation is associated with a deletion of all or part of the amino acid sequence set forth in SEQ ID NO: 19. More preferably, the aberrant K4 polypeptide comprises the sequence set forth in SEQ ID NO: 15.

In a further aspect, the invention provides an antigen-binding molecule that is immuno-interactive specifically with an isolated aberrant K4 polypeptide as broadly described above.

The invention also encompasses the use of an isolated polynucleotide as broadly described above, the use of a probe as broadly described above, the use of an isolated polypeptide as broadly described above or the use of an antigen-binding molecule as broadly described above for detecting an aberrant KLK4 polynucleotide, or an aberrant K4 polypeptide that correlate with at least one condition selected from a cancer or a benign tumour.

### BRIEF DESCRIPTION OF THE DRAWINGS

FIG. 1: RT-PCR of KLK4 (upper panel) and P2-microglobulin (lower panel) in a number of endometrial cancer cell lines, HA (HEC1A), HB (HEC1B), ISH (Ishikawa), KLE, and RL (RL95-2). Controls include a negative (−VE, no cDNA), LNC (LNCaP) and K (Kidney). The sizes of the molecular weight marker (marker IV, Roche) and PCR products, in base pairs, are indicated at left and right respectively.

FIG. 2: Southern analysis of the ethidium bromide gel from FIG. 1. The upper panel was probed with an exon 3 probe resulting in two bands of 526 and 389 bp. The lower panel was probed with an exon 4 probe resulting in a single product of 526 bp.

FIG. 3: Protein sequence of the KLK4 wild type (upper lines) and exon 4 deleted predicted product (lower lines). The five exons are marked EX1-EX5 and the exon junctions are indicated by an arrow (i). The exon 4 deletion is indicated by a dashed line (-----). The catalytic triad, Histidine (H), Aspartic acid (D) and Serine (S), essential for catalytic activity, are marked in bold. The asterisk indicates the end of the protein sequence. For the exon 4 deleted form, this is a glycine, preceding a premature stop codon.

FIG. 4: Western analysis of K4 in endometrial cancer cell lines. The endometrial cancer cell lines are as noted for FIG. 1, except for the last lane PT (prostate tissue). High levels of K4 intracellular protein were observed in most lines except for two moderately differentiated lines, HEC1A (HA) and RL95-2 (RL). K4 protein was observed at approximately 38-40 kDa.

FIG. 5: K4 western analysis of 200 μg intracellular protein from the KLE cell line treated with 10 nmol/L estradiol and progesterone for 48 h. Lanes: C (control, no treatment), E (estradiol), P (progesterone) and E/P (estradiol for 24 h followed by 24 h treatment with estradiol and progesterone).

FIG. 6: KLK4 expression in normal ovaries and ovarian tumours. A. Southern blot analysis of the KLK4 RT-PCR products with the DIG-labelled exon 3 KLK4 probe. B. Ethidium bromide stained agarose gel of the RT-PCR for P2-microglobulin as an internal control. Lanes 1-3, normal ovarian tissues; Lanes 4-6, normal ovarian epithelial cells; Lanes 7-8, primary cultured cells from serous adenomas of ovary (BNG: benign); Lanes 9-10, primary cultured cells from stage II serous carcinomas of ovary; Lanes 11-13, primary cultured cells from stage III and IV serous carcinomas of ovary; Lanes 14-16, serous ovarian carcinoma tissues; Lanes 17-18, granulosa cell tumour tissues (GCT); Lanes 19-20, mucinous adenoma, mucinous carcinoma tissues (MUC); Lanes 21-22, Serous ovarian carcinoma cell lines: OVCAR-3, and OAW42; Lane 23, prostate cancer cell line LNCaP and Lane 24, negative control (no cDNA). The tumour cells marked with * were used for DNA sequencing analysis of the alternate spliced forms.

FIG. 7: KLK4 mRNA variant expression in normal ovaries and ovarian tumours. A. Ethidium bromide stained agarose gel of the RT-PCR for KLK4 with exon 2 and exon 5 PCR primers. Lane 1, NOE; Lane 2, Primary cultured serous ovarian carcinoma cells (SER Ca); Lane 3, Ovarian carcinoma cell line OAW42; Lane 4, LNCaP as positive control; Lane 5, Negative control (no cDNA). B. RT-PCR for β₂-microglobulin as an internal control. The sizes of the variant and wild type PCR products are indicated to the right. DNA sequencing was performed on the PCR products marked *. C. Amino acid sequence of the KLK4 putative product from the wild type and three variants. The five exons of the coding region are marked and the introns are indicated by a dot line ( . . . ). The intronic insertion (intron 3) is indicated by underline (_{———}). The exon 4 deletion is indicated as (---). The amino acids that constitute the catalytic triad, Histidine (H), Aspartic acid (D) and Serine (S), are marked in bold. The asterisk indicates the end of the predicted protein sequence.

FIG. 8: KLK4 mRNA and its protein expression in ovarian cancer. A. Well differentiated serous ovarian carcinoma showing KLK4 mRNA transcript expression (arrows), as detected by in situ hybridisation with DIG-labelled antisense KLK4 cRNA probe. Scale bar=40 μm. S=stroma. Ca=cancer. B. Hybridisation with the DIG-labelled KLK4 sense cRNA probe as the negative control. Scale bar=80 μm. C. Well differentiated serous ovarian carcinoma showing K4 cytoplasmic and membrane expression (arrows), as detected by affinity purified anti-peptide K4 antibody. Scale bar=200 μm. S=stroma. Ca=cancer cells. D. Normal serum used as the negative control. Scale bar=60 μm. E. Western blot analysis with and an affinity purified K4 antibody of cytoplasmic extract (˜150 μg protein) from the ovarian cancer cell line (OAW42), primary cultured serous ovarian carcinoma cells (N12, N15), prostate cancer cell line LNCaP and β-estradiol treatment on the ovarian cancer cell line OVCAR-3. The size of the protein molecular weight markers is shown at the left. A K4 protein of ≈Mᵣ 40,000 was observed. F. Densitometry of the above Western blot, showing the up-regulation of the intracellular hK4 levels following β-estradiol treatment (100 nM) on OVCAR-3 over 30 hrs.

FIG. 9: Western blot analysis of cytoplasmic (cyto) extracts (˜150 μg protein) and nuclear (Nu) extracts (−20 μg protein) obtained from normal ovarian epithelial cells (NOE), from the ovarian cancer cell line OVCAR-3 and serous ovarian cancer cells (Ser Ca) using an antibody specific to a C-terminal portion of K4. This figure shows differential expression of K4 in cytoplasmic and nuclear extracts of cultured cells. A high molecular weight K4-containing species (HMWK4) and a low molecular weight K4-containing species (LMWK4) is present in all cytoplasmic extracts tested. A medium molecular weight K4-containing species (MMWK4) and/or a low molecular weight K4-containing species (LMWK4) is present in all nuclear extracts tested. The nuclear extract of NOE primarily contained MMWK4, whereas the nuclear extract of OVCAR-3 contains both MMWK4 and LMWK4 and the nuclear extract of SER Ca predominantly contains LMWK4.

FIG. 10: Immunohistochemistry with specific K4 antibody (C-terminal) showing nuclear staining in both normal and cancer glands of prostatic tissue. The negative control was performed with no addition of primary antibody. Cancer glands (C) and normal glands (N) of the prostate showing positive staining in the nucleus ( [symbol] ).

FIG. 11: Immunohistochemical staining with an anti N-terminal peptide K4 antibody hK4 showing distinct nuclear staining of malignant cells of high grade PIN lesion (arrow head). Note the nucleus of basal cells of PIN represent negative staining (arrow).

### BRIEF DESCRIPTION OF THE SEQUENCES: SUMMARY TABLE

TABLE A

| SEQUENCE ID | DESCRIPTION | LENGTH |
|---|---|---|
| SEQ ID NO: 1 | Nucleotide sequence corresponding to an alternately | 848 nts |
| | spliced variant of human KLK4, comprising the intronic | |
| | sequence between exons 3 and 4 of normal KLK4 | |
| SEQ ID NO: 2 | Aberrant K4 polypeptide encoded by SEQ ID NO: 1 | 195 aa |
| SEQ ID NO: 3 | Nucleotide sequence corresponding to another alternately | 628 nts |
| | spliced variant of human KLK4 mRNA, which excludes | |
| | coding sequence corresponding to exon 4 of normal KLK4 | |
| SEQ ID NO: 4 | Aberrant K4 polypeptide encoded by SEQ ID NO: 3 | 159 aa |
| SEQ ID NO: 5 | Nucleotide sequence corresponding to normal KLK4 | 765 nts |
| | mRNA as set forth in GenBank Accession No. AF148532 | |
| SEQ ID NO: 6. | Normal K4 polypeptide encoded by SEQ ID NO: 5 | 254 aa |
| SEQ ID NO: 7 | Nucleotide sequence corresponding to the intronic | 83 nts |
| | sequence of SEQ ID NO: 1 | |
| SEQ ID NO: 8 | Nucleotide sequence corresponding to a 3′ portion of SEQ | 110 nts |
| | ID NO: 1 | |
| SEQ ID NO: 9 | Polypeptide product encoded by SEQ ID NO: 8 | 36 aa |
| SEQ ID NO: 10 | Nucleotide sequence corresponding to exon 4 deletion in | 137 nts |
| | SEQ ID NO: 3 | |
| SEQ ID NO: 11 | Amino acid sequence encoded by SEQ ID NO: 10 | 45 aa |
| SEQ ID NO: 12 | Complete nucleotide sequence of the KLK4 gene as set | 4385 nts |
| | forth in GenBank Accession No. AF148532 | |
| SEQ ID NO: 13 | Polypeptide encoded by SEQ ID NO: 12 | 254 aa |
| SEQ ID NO: 14 | Nucleotide sequence corresponding to another alternately | 640 nts |
| | spliced variant of human KLK4 mRNA, which includes | |
| | sequence corresponding to intron 2 of KLK4 and which | |
| | excludes coding sequence corresponding to exon 4 of | |
| | KLK4 | |
| SEQ ID NO: 15 | Aberrant K4 polypeptide encoded by SEQ ID NO: 14 | 75 aa |
| SEQ ID NO: 16 | Nucleotide sequence corresponding to the intronic | 12 nts |
| | sequence of SEQ ID NO: 14 | |
| SEQ ID NO: 17 | Nucleotide sequence corresponding to the intron between | 421 nts |
| | exons 2 and 3 of KLK4 | |
| SEQ ID NO: 18 | Nucleotide sequence corresponding to exon 3-exon 4 | 404 nts |
| | splice variant of KLK4 | |
| SEQ ID NO: 19 | K4 amino acid sequence deleted in the aberrant | 180 aa |
| | polypeptide of SEQ ID NO: 15 | |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “aberrant polynucleotide” refers to a polynucleotide resulting from a substitution, deletion and/or addition of one or more nucleotides in a “normal” reference polynucleotide and which correlates with the presence or risk of a cancer or benign tumour.

The terms “aberrant polynucleotide variant” and “variant” refer to polynucleotides displaying substantial sequence identity with a reference aberrant polynucleotide sequence or polynucleotides that hybridise with a reference aberrant polynucleotide sequence under stringent conditions that are defined hereinafter, wherein the variant polynucleotides comprise the same alteration as the reference aberrant polynucleotide sequence, or an alteration that encodes the same aberrant amino acid(s) (silent alteration) encoded by the reference polynucleotide sequence. The terms “aberrant polynucleotide variant” and “variant” also include naturally occurring allelic variants.

The term “aberrant polypeptide” refers to a polypeptide resulting from a substitution, deletion and/or addition of one or more amino acid residues in a “normal” reference polypeptide and which correlates with the presence or risk of a cancer or benign tumour.

The term “aberrant polypeptide variant” refers to aberrant polypeptides which are distinguished from a normal polypeptide by the addition, deletion or substitution of at least one amino acid but otherwise comprise the same aberration which correlates with the presence or risk of a cancer or benign tumour. In this regard, it is well understood in the art that some amino acids may be changed to others with broadly similar properties without changing the nature of the activity of the aberrant polypeptide (conservative substitutions) as described hereinafter. Accordingly, the present invention encompasses immuno-mimetic polypeptides that can elicit the production of elements that are immuno-interactive with a naturally occurring aberrant K4 polypeptide.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Antigenic or immunogenic activity” refers to the ability of a polypeptide, fragment, variant or derivative according to the invention to produce an antigenic or immunogenic response in a mammal to which it is administered, wherein the response includes the production of elements which specifically bind the polypeptide or fragment thereof.

The term “biological sample” as used herein refers to a sample that may be extracted, untreated, treated, diluted or concentrated from a patient. Suitably, the biological sample is selected from tissue samples including tissue from the ovaries, endometrium, and prostate.

By “biologically active fragment” is meant a fragment of a full-length parent polypeptide which fragment retains the activity of the parent polypeptide. A biologically active fragment will therefore have, for example, the serine protease activity of K4 or the ability to elicit the production of elements that specifically bind to K4. As used herein, the term “biologically active fragment” includes deletion variants and small peptides, for example of at least 10, preferably at least 20 and more preferably at least 30 contiguous amino acids, which comprise the above activities. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

By “effective amount”, in the context of treating or preventing a condition is meant the administration of that amount of active substance to an individual in need of such treatment or prophylaxis, either in a single dose or as part of a series, that is effective for treatment or prophylaxis of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the terms “function” “functional” and the like refer to a biological, enzymatic, or therapeutic function.

By “functional KLK4 polynucleotide” or “functional K4 polypeptide” is meant a KLK4 polynucleotide or K4 polypeptide having no structural or functional defects which correlate with at least one condition selected from a cancer or a benign tumour.

“Homology” refers to the percentage number of amino acids that are identical or constitute conservative substitutions as defined in Table B below. Homology may be determined using sequence comparison programs such as GAP (Deveraux et al. 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

Reference herein to “immuno-interactive” includes reference to any interaction, reaction, or other form of association between molecules and in particular where one of the molecules is, or mimics, a component of the immune system.

By “immuno-interactive fragment” is meant a fragment of a polypeptide of the invention as for example set forth in SEQ ID NO: 2, 4 or 15, which fragment elicits an immune response, including the production of elements that specifically bind to said polypeptide, or variant or derivative thereof. As used herein, the term “immuno-interactive fragment” includes deletion variants and small peptides, for example of at least six, preferably at least 8 and more preferably at least 20 contiguous amino acids, which comprise antigenic determinants or epitopes. Several such fragments may be joined together.

By “isolated” is meant material that is substantially or essentially free from components that normally accompany it in its native state.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linking” a promoter to a polynucleotide is meant placing the polynucleotide (e.g., protein encoding polynucleotide or other transcript) under the regulatory control of a promoter, which then controls the transcription and optionally translation of that polynucleotide. In the construction of heterologous promoter/structural gene combinations, it is generally preferred to position a promoter or variant thereof at a distance from the transcription start site of the polynucleotide, which is approximately the same as the distance between that promoter and the gene it controls in its natural setting; i.e.: the gene from which the promoter is derived. As is known in the art, some variation in this distance can be accommodated without loss of function.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes, avians, reptiles).

By “pharmaceutically acceptable carrier” is meant a solid or liquid filler, diluent or encapsulating substance that can be safely used in topical or systemic administration to a animal, preferably a mammal including humans.

The term “polynucleotide” or “nucleic acid” as used herein designates mRNA, RNA, cRNA, cDNA or DNA. The term typically refers to oligonucleotides greater than 30 nucleotide residues in length.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but can alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridise and serve as a site for the initiation of synthesis. By “substantially complementary”, it is meant that the primer is sufficiently complementary to hybridise with a target polynucleotide. Preferably, the primer contains no mismatches with the template to which it is designed to hybridise but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridisation conditions. Probes can be labelled directly or indirectly.

The term “recombinant polynucleotide” as used herein refers to a polynucleotide formed in vitro by the manipulation of a polynucleotide into a form not normally found in nature. For example, the recombinant polynucleotide can be in the form of an expression vector. Generally, such expression vectors include transcriptional and translational regulatory polynucleotide operably linked to the polynucleotide.

By “recombinant polypeptide” is meant a polypeptide made using recombinant techniques, ie. through the expression of a recombinant or synthetic polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (ie. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (ie. resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (eg. A, T, C, G, I) or the identical amino acid residue (eg. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (ie. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation and washing procedures. The higher the stringency, the higher will be the degree of complementarity between immobilised target nucleotide sequences and the labelled probe polynucleotide sequences that remain hybridised to the target after washing.

“Stringent conditions” refers to temperature and ionic conditions under which only nucleotide sequences having a high frequency of complementary bases will hybridise. The stringency required is nucleotide sequence dependent and depends upon the various components present during hybridisation and subsequent washes, and the time allowed for these processes. Generally, in order to maximise the hybridisation rate, non-stringent hybridisation conditions are selected; about 20 to 25° C. lower than the thermal melting point (Tₘ). The Tₘ is the temperature at which 50% of specific target sequence hybridises to a perfectly complementary probe in solution at a defined ionic strength and pH. Generally, in order to require at least about 85% nucleotide complementarity of hybridised sequences, highly stringent washing conditions are selected to be about 5 to 15° C. lower than the Tₘ. In order to require at least about 70% nucleotide complementarity of hybridised sequences, moderately stringent washing conditions are selected to be about 15 to 30° C. lower than the Tₘ. Highly permissive (low stringency) washing conditions may be as low as 50° C. below the Tₘ, allowing a high level of mismatching between hybridised sequences. Those skilled in the art will recognise that other physical and chemical parameters in the hybridisation and wash stages can also be altered to affect the outcome of a detectable hybridisation signal from a specific level of homology between target and probe sequences.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, ie. a vector that exists as an extrachromosomal entity, the replication of which is independent of chromosomal replication, eg. a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

As used herein, underscoring or italicising the name of a gene shall indicate the gene, in contrast to its protein product, which is indicated in the absence of any underscoring or italicising. For example, “KLK4” shall mean the KLK4 gene, whereas “K4” shall indicate the protein product of the “KLK4” gene.

2. Polynucleotides

2.1 Aberrant K4 Polynucleotides

The present invention is predicated in part on the determination that alterations, inclusive of substitutions, deletions and additions, within the coding regions of KLK4, correlate with the presence or risk of a cancer or a benign tumour and possibly of other conditions related to aberrant KLK expression such as brain disorders including Alzheimer's disease. The invention, therefore, provides in one aspect an isolated polynucleotide comprising a nucleotide sequence which corresponds or is complementary to at least a portion of an aberrant KLK4 polynucleotide that correlates with the presence or risk of at least one condition selected from a cancer or a benign tumour. The aberrant KLK4 polynucleotide may be distinguished from a normal KLK4 polynucleotide by the substitution, deletion or addition of at least one nucleotide. Such substitution, addition or deletion can reside anywhere in KLK4 or transcript thereof, and preferably in an open reading frame of KLK4. In a preferred embodiment, the aberrant KLK4 polynucleotide encodes a polypeptide having altered, impaired or abrogated function relative to a normal K4 polypeptide.

Preferably, the aberrant KLK4 polynucleotide is selected from the group consisting of: (a) a polynucleotide comprising the entire sequence of nucleotides set forth in SEQ ID NO: 1; (b) a polynucleotide fragment of (a), wherein said fragment comprises SEQ ID NO: 7 or fragment thereof; (c) a polynucleotide comprising the entire sequence of nucleotides set forth in SEQ ID NO: 3; and (d) a polynucleotide fragment of (c), wherein said fragment comprises the codon spanning nucleotide 475 through 477 of SEQ ID NO: 3.

As will be more fully described hereinafter, SEQ ID NO: 1 corresponds to an alternately spliced variant of KLK4, comprising essentially the entire intronic sequence (i.e., intron 3) located between exons 3 and 4 of the normal or wild-type human KLK4 gene, as for example set forth in SEQ ID NO: 12. More particularly, SEQ ID NO: 1 has an 83 bp insertion of intronic sequence from intron 3, which causes a frame shift of the coding region that yields a premature stop codon and which thereby gives rise to a truncated protein product (about 59 amino acids smaller compared to wild type K4) that does not contain the serine residue (Ser²⁰⁷) of the catalytic triad as for example contained in SEQ ID NO: 6. Thus, the translated product of the splice variant has an altered carboxyl terminal portion. The deduced amino acid sequence of this carboxyl terminal portion and its corresponding nucleotide sequence are set presented in SEQ ID NO: 9 and 8, respectively.

SEQ ID NO: 3 corresponds to another alternately spliced variant of KLK4, but in this instance, comprises a deletion of a region corresponding to exon 4 of normal KLK4 as set forth in SEQ ID NO: 12. The region deleted in SEQ ID NO: 3, relative to normal KLK4, consists essentially of the sequence set forth in SEQ ID NO: 10 and results in a truncated polypeptide, which is about 95 amino acids smaller than wild type K4. More particularly, the variant (SEQ ID NO: 3) consists of 4 exons, (instead of 5) with exon 3 joining to exon 5 thus altering the reading frame. This results in a stop codon encoded by the second codon of exon 5. As this alternatively spliced transcript does not possess the essential serine residue required for catalytic activity, it is unlikely it will encode a functional enzyme.

SEQ ID NO: 14 corresponds to yet another alternately spliced variant of KLK4, comprising an insertion of 12 nucleotides of a region corresponding to intron 2 of normal KLK4 as set forth in SEQ ID NO: 12. The region inserted in SEQ ID NO: 14, relative to normal KLK4, consists essentially of the sequence set forth in SEQ ID NO: 16. SEQ ID NO: 14 also comprises a deletion of a region corresponding to exon 4 of normal KLK4 as set forth in SEQ ID NO: 12. The region deleted in SEQ ID NO: 14, is essentially the same as that deleted in respect of SEQ ID NO: 3 above, and results in a truncated polypeptide, which is 179 amino acids smaller than wild type K4. More particularly, the variant (SEQ ID NO: 14) consists of 4 exons, (instead of 5) with the intronic insertion, immediately downstream of exon 2, joining to exon 3. The misplicing of exon 3 to the intronic sequence results in an alteration to the coding sequence. The final codon of exon 2 changes from an Asn to Lys, and a stop codon is introduced immediately downstream of the altered codon.

Alternatively, the aberrant KLK4 polynucleotide may comprise an N-terminal truncation as for example described by Korkmaz et al. (2001, DNA and Cell Biology. 20(7): 435-445).

K4 shares 78% homology at the amino acid level with a pig enamel matrix serine protease (EMSP1) which is involved in the degradation of the extracellular matrix (ECM) in preparation of enamel maturation (14, 15). Two other family members of the KLK family, KLK6 (zyme/protease M/neurosin) (7) and KLK7 (stratum corneum chymotryptic enzyme, SCCE) (16) respectively, have also been implicated in ECM remodelling in cancer. SCCE is a skin-specific serine protease involved in the degradation of intracellular cohesive structures in the continuous shedding of skin cells (16), and protease M is thought to be involved in the development of primary breast and ovarian tumours (7). Not wishing to be bound by any one particular theory or mode of action, it is possible that KLK4 may also play a similar role in ECM degradation and contribute to the pathophysiological processes of cancer.

However, it will be understood that the invention contemplates any isolated aberrant KLK4 that correlates with the presence or risk of a cancer or a benign tumour, other than those comprising the above insertions and deletions. Such aberrant KLK4 polynucleotides may be obtained from individuals afflicted with a cancer or a benign tumour. In a preferred embodiment, the cancer or benign tumour is associated with an organ or tissue selected from the group consisting of ovaries, endometrium and prostate.

Nucleic acid isolation protocols are well known to those of skill in the art. For example, aberrant KLK4 polynucleotide may be prepared according to the following procedure:

(a) creating primers which are optionally degenerate wherein each comprises a portion of a normal KLK4 polynucleotide, preferably encoding the sequence set forth in SEQ ID NO: 6;

(b) obtaining a nucleic acid extract from an individual affected with at least one of said conditions; and

(c) using said primers to amplify, via nucleic acid amplification techniques, at least one amplification product from said nucleic acid extract, wherein said amplification product corresponds to an aberrant KLK4 polynucleotide or fragment thereof.

Suitable nucleic acid amplification techniques are well known to the skilled addressee, and include polymerase chain reaction (PCR) as for example described in Ausubel et al (supra); strand displacement amplification (SDA) as for example described in U.S. Pat. No. 5,422,252; rolling circle replication (RCR) as for example described in Liu et al., (1996, J. Am. Chem. Soc. 118:1587-1594 and International application WO 92/01813) and Lizardi et al., (International Application WO 97/19193); nucleic acid sequence-based amplification (NASBA) as for example described by Sooknanan et al, (1994, Biotechniques 17:1077-1080); and Q-0 replicase amplification as for example described by Tyagi et al, (1996, Proc. Natl. Acad. Sci. USA 93: 5395-5400).

2.2 Aberrant Polynucleotide Variants

The present invention also encompasses aberrant polynucleotide variants displaying substantial sequence identity with a reference aberrant KLK4 polynucleotide which correlates with the presence or risk of a cancer or a benign tumour, wherein the variant polynucleotides comprise the same alteration as the reference aberrant polynucleotide, or an alteration that encodes the same aberrant amino acid(s) (silent alteration) encoded by the reference polynucleotide. Also encompassed are aberrant polynucleotide variants that hybridise with a reference aberrant KLK4 polynucleotide sequence under stringent conditions that are defined hereinafter. Practitioners in the art will recognise that in view of the degeneracy in the genetic code, silent alterations to a reference aberrant polynucleotide can be made to provide a synonymous polynucleotide encoding the same polypeptide as the reference aberrant polynucleotide. Also encompassed are aberrant polynucleotide variants that hybridise with a reference aberrant KLK4 polynucleotide sequence under stringent conditions that are defined hereinafter.

Typically, polynucleotide variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridising the membrane-bound DNA to a complementary nucleotide sequence labelled radioactively, enzymatically or fluorochromatically. In dot blotting and slot blotting, DNA samples are directly applied to a synthetic membrane prior to hybridisation as above.

An alternative blotting step is used when identifying complementary polynucleotides in a cDNA or genomic DNA library, such as through the process of plaque or colony hybridisation. A typical example of this procedure is described in Sambrook et al. (“Molecular Cloning. A Laboratory Manual”, Cold Spring Harbour Press, 1989, Chapters 8-12).

Typically, the following general procedure can be used to determine hybridisation conditions. Polynucleotides are blotted/transferred to a synthetic membrane, as described above. A reference polynucleotide such as a polynucleotide of the invention is labelled as described above, and the ability of this labelled polynucleotide to hybridise with an immobilised polynucleotide is analysed.

A skilled addressee will recognise that a number of factors influence hybridisation. The specific activity of radioactively labelled polynucleotide sequence should typically be greater than or equal to about 10⁸ dpm/mg to provide a detectable signal. A radiolabelled nucleotide sequence of specific activity 10⁸ to 10⁹ dpm/mg can detect approximately 0.5 pg of DNA. It is well known in the art that sufficient DNA must be immobilised on the membrane to permit detection. It is desirable to have excess immobilised DNA, usually 10 μg. Adding an inert polymer such as 10% (w/v) dextran sulfate (MW 500,000) or polyethylene glycol 6000 during hybridisation can also increase the sensitivity of hybridisation (see Ausubel supra at 2.10.10).

To achieve meaningful results from hybridisation between a polynucleotide immobilised on a membrane and a labelled polynucleotide, a sufficient amount of the labelled polynucleotide must be hybridised to the immobilised polynucleotide following washing. Washing ensures that the labelled polynucleotide is hybridised only to the immobilised polynucleotide with a desired degree of complementarity to the labelled polynucleotide.

It will be understood that polynucleotide variants according to the invention will hybridise to a reference polynucleotide under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridise to a reference polynucleotide under high stringency conditions. High stringency conditions include and encompass from at least about 31% v/v to at least about 50% v/v formamide and from about 0.01 M to about 0.15 M salt for hybridisation at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:

Tₘ=81.5+16.6 (log₁₀ M)+0.41 (% G+C)−0.63 (% formamide)−(600/length)

wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In a preferred hybridisation procedure, a membrane (e.g. a nitrocellulose membrane or a nylon membrane) containing immobilised DNA is hybridised overnight at 42° C. in a hybridisation buffer (50% deionised formamide, 5×SSC, 5× Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (ie., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

Methods for detecting a labelled polynucleotide hybridised to an immobilised polynucleotide are well known to practitioners in the art. Such methods include autoradiography, phosphorimaging, and chemiluminescent, fluorescent and calorimetric detection.

3. Vectors

The invention also contemplates a vector comprising a polynucleotide according to the invention. Vectors contemplated by the present invention include cloning vectors and expression vectors.

In one embodiment, a polynucleotide of the invention is suitably rendered expressible in a host cell by operably linking the polynucleotide with a regulatory polynucleotide. The synthetic construct or vector thus produced may be introduced firstly into an organism or part thereof before subsequent expression of the construct in a particular cell or tissue type. Any suitable organism is contemplated by the invention, which may include unicellular as well as multi-cellular organisms. Suitable unicellular organisms include bacteria. Exemplary multi-cellular organisms include yeast, mammals and plants.

The construction of the vector may be effected by any suitable technique as for example described in the relevant sections of Ausubel et al. (supra) and Sambrook et al. (supra). However, it should be noted that the present invention is not dependent on and not directed to any one particular technique for constructing the vector.

Regulatory polynucleotides which may be utilised to regulate expression of the polynucleotide include, but are not limited to, a promoter, an enhancer, and a transcriptional terminator. Such regulatory sequences are well known to those of skill in the art. Suitable promoters that may be utilised to induce expression of the polynucleotides of the invention include constitutive promoters and inducible promoters.

4. Host Cells and Cell Lines

The invention also encompasses a host cell comprising a vector as broadly described above, as well as a cell line that comprises a polynucleotide comprising a nucleotide sequence which corresponds or is complementary to at least a portion of an aberrant KLK4 polynucleotide that correlates with the presence or risk of at least one condition selected from a cancer or a benign tumour. The cell line is preferably produced from a cell of an individual, which cell an aberrant KLK4 polynucleotide that has said correlation. Many methods of producing the cell line are known to those of skill in the art. Suitably, the cell line is obtained by immortalisation of a cell with Epstein-Barr virus as is known in the art.

5. Polypeptides

5.1 Aberrant K4 Polypeptides

The present invention also encompasses an isolated polypeptide comprising an amino acid sequence which corresponds to at least a portion of an aberrant CBG polypeptide that correlates with the presence or risk of at least one condition selected from a cancer or a benign tumour. The aberrant K4 polypeptide may result from a mutation to a KLK4 genetic sequence, an alternate splicing event, an aberrant post-transcriptional modification or an aberrant post-translational modification. The aberrant K4 polypeptide preferably has impaired, altered or abrogated function relative to normal K4.

The invention contemplates full-length aberrant K4 polypeptides as well as fragments, variants and derivatives of these comprising the aberration (e.g., mutation) corresponding to a reference aberrant K4 polypeptide. The aberration suitably corresponds to an addition, deletion or substitution of one or more residues relative to normal K4. In a preferred embodiment, the sequence aberration corresponds to an insertion, which preferably comprises all or part of the sequence set forth in SEQ ID NO: 9. In this instance, the aberrant K4 polypeptide preferably comprises the sequence set forth in SEQ ID NO: 2. In another embodiment, the sequence aberration corresponds to a truncation or deletion, which preferably consists of all or part of the sequence set forth in SEQ ID NO: 11. In this instance, the aberrant K4 polypeptide preferably comprises the sequence set forth in SEQ ID NO: 4. In yet another embodiment, the sequence aberration corresponds to a truncation or deletion, which preferably consists of all or part of the sequence set forth in SEQ ID NO: 10. In this instance, the aberrant K4 polypeptide preferably comprises the sequence set forth in SEQ ID NO: 15.

5.2 Aberrant Polypeptide Variants

With regard to variant polypeptides of the invention, it will be understood that such variants should retain antigenic or immunogenic activity of the parent or reference aberrant polypeptide, which includes the production of elements that specifically bind to the amino acid sequence which corresponds to at least a portion of an aberrant K4 polypeptide that correlates with the presence or risk of a cancer or a benign tumour. Such variant polypeptides, therefore, constitute immuno-mimetics, which mimic the immunogenicity or antigenicty of a reference variant polypeptide. Exemplary conservative substitutions in a parent polypeptide mutant may be made according to TABLE B:

TABLE B

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE B. Other replacements would be non-conservative substitutions and relatively fewer of these may be tolerated. Generally, the substitutions which are likely to produce the greatest changes in a polypeptide's properties are those in which (a) a hydrophilic residue (eg. Ser or Thr) is substituted for, or by, a hydrophobic residue (eg. Ala, Leu, Ile, Phe or Val); (b) a cysteine or proline is substituted for, or by, any other residue; (c) a residue having an electropositive side chain (eg. Arg, His or Lys) is substituted for, or by, an electronegative residue (eg. Glu or Asp) or (d) a residue having a bulky side chain (eg. Phe or Trp) is substituted for, or by, one having a smaller side chain (eg. Ala, Ser) or no side chain (eg. Gly).

In general, variants comprise regions that are at least 75% homologous, more suitably at least 80%, preferably at least 85%, and most preferably at least 90% homologous to the basic sequences as for example shown in SEQ ID NO: 2, 4 or 15. In an alternate embodiment, variants comprise regions that have at least 60%, more suitably at least 70%, preferably at least 80%, and most preferably at least 90% identity over a parent amino acid sequence of identical size (“comparison window”) or when compared to an aligned sequence in which the alignment is performed by a computer homology program known in the art. What constitutes suitable variants may be determined by conventional techniques. For example, nucleic acids encoding polypeptides according to SEQ ID NO: 2, 4 or 15 can be mutated using either random mutagenesis for example using transposon mutagenesis, or site-directed mutagenesis. The resultant DNA fragments are then cloned into suitable expression hosts such as E. coli using conventional technology and clones that retain the desired activity are detected. For example, the desired activity may include antigenic or immunogenic activity resulting in the production of elements that specifically bind the parent aberrant K4 polypeptide linked to a condition referred to above. Where the clones have been derived using random mutagenesis techniques, positive clones would have to be sequenced in order to detect the mutation.

5.3 Aberrant Polypeptide Derivatives

Suitable derivatives of K4 include amino acid deletions and/or additions to an aberrant K4 polypeptide according to the invention such as but not limited to SEQ ID NO: 2, 4 or 15, or variants thereof, wherein said derivatives retain antigenic or immunogenic activity, which includes the production of elements that specifically bind to the parent polypeptide. “Additions” of amino acids may include fusion of the aberrant polypeptides, fragments thereof or variants of these with other polypeptides or proteins. In this regard, it will be appreciated that the aberrant polypeptides, aberrant polypeptide fragments or variants of the invention may be incorporated into larger polypeptides, and such larger polypeptides may also be expected to retain the antigenic or immunogenic activity mentioned above.

The aberrant polypeptides according to the invention, fragments thereof or variants of these may be fused to a further protein, for example, which is not derived from the original host. The other protein may, by way of example, assist in the purification of the protein. For instance a polyhistidine tag or a maltose binding protein may be used in this respect as described in more detail below. Alternatively, it may produce an antigenic response or immunogenic response that is effective against the aberrant polypeptide or fragment thereof. Other possible fusion proteins are those which produce an immunomodulatory response. Particular examples of such proteins include Protein A or glutathione S-transferase (GST). In addition, the aberrant polypeptide, fragment thereof or variant of these may be fused to an oligosaccharide based vaccine component where it acts as a carrier protein.

The invention also contemplates fragments of the aberrant polypeptide of the invention. In one embodiment, polypeptides consisting of or comprising a fragment of an aberrant K4 polypeptide consisting of at least 10 contiguous amino acids of an aberrant K4 polypeptide is provided, wherein said fragment comprises a sequence aberration that correlates with a condition mentioned above. In other embodiments, the fragment consists of at least 5, preferably at least 10, more preferably at least 20 and even more preferably at least 50 contiguous amino acids of an aberrant K4 polypeptide, wherein said fragment comprises a sequence aberration that correlates with said condition. For example, the fragment may comprise all or part of the sequence set forth in SEQ ID NO: 9 corresponding to the carboxyl terminal sequence of SEQ ID NO: 2, which varies significantly from the normal K4 carboxyl terminal sequence. Alternatively, the fragment may comprise a portion of the sequence set forth in SEQ ID NO: 15, said portion comprising a lysine residue, which replaces Asn⁷⁵ of wild-type K4.

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention.

Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2, 4, 6-trinitrobenzene sulphonic acid (TNBS).

The carboxyl group may be modified by carbodiimide activation via O-acylisourea formation followed by subsequent derivatisation, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in TABLE C.

TABLE C

| Non-conventional amino acid | Non-conventional amino acid |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

Polypeptides of the inventions may be prepared by any suitable procedure known to those of skill in the art. For example, the polypeptides may be prepared by a procedure including the steps of:

(a) preparing a recombinant polynucleotide containing a nucleotide sequence encoding an aberrant K4 polypeptide (eg. SEQ D NO: 2, 4 or 15 or fragment thereof, or variant or derivative of these), which nucleotide sequence is operably linked to a regulatory polynucleotide;

(b) introducing the recombinant polynucleotide into a suitable host cell;

(c) culturing the host cell to express recombinant polypeptide from said recombinant polynucleotide; and

(d) isolating the recombinant polypeptide.

Suitably, said nucleotide sequence comprises SEQ ID NO: 1, 3 or 14.

The recombinant polynucleotide is preferably in the form of an expression vector that may be either a self-replicating extra-chromosomal vector such as a plasmid, or a vector that integrates into a host genome.

The regulatory polynucleotide may comprise transcriptional and translational regulatory nucleic acid that will generally be appropriate for the host cell used for expression. Numerous types of appropriate expression vectors and suitable regulatory sequences are known in the art for a variety of host cells. Typically, the transcriptional and translational regulatory nucleic acid may include, but is not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide. Well known examples of fusion partners include, but are not limited to, glutathione-S-transferase (GST), Fc portion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG.

Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application. Preferably, the fusion partners also have protease cleavage sites, such as for Factor Xₐ or Thrombin, which allow the relevant protease to partially digest the fusion polypeptide of the invention- and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be effected by any suitable method including transfection, and transformation, the choice of which will be dependent on the host cell employed. Such methods are well known to those of skill in the art. Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

Alternatively, the aberrant polypeptides, aberrant polypeptide fragments and variants or derivatives of the invention may be synthesised using solution synthesis or solid phase synthesis as described, for example, in Chapter 9 of Atherton and Shephard (supra) and in Roberge et al (1995, Science 269: 202).

6. Antigen-Binding Molecules

The invention also contemplates antigen-binding molecules that bind specifically to the aforementioned aberrant polypeptides, aberrant polypeptide fragments, variants and derivatives. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Köhler and Milstein (1975, Nature 256, 495-497), or by more recent modifications thereof as described, for example, in Coligan et al., (1991, supra) by immortalising spleen or other antibody-producing cells derived from a production species which has been inoculated with one or more of the aberrant polypeptides, aberrant polypeptide fragments, variants or derivatives of the invention. Exemplary monoclonal antibodies have been generated against an N-terminal portion (INGEDCSPHSQ; residues 31 through 41), a central portion (LSVRHPEYNRPLL; residues 101 through 113), and a C-terminal portion (SEEVCSKLYDPLYHPS; residues 174 through 189), of wild-type K4.

The invention also contemplates as antigen-binding molecules Fv, Fab, Fab′ and F(ab′)₂ immunoglobulin fragments. Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whore antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al (Kreber et al. 1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plünckthun et al (1996, In Antibody engineering: A practical approach. 203-252).

Alternatively, the synthetic stabilised Fv fragment comprises a disulphide stabilised Fv (dsFv) in which cysteine residues are introduced into the V_{H} and V_{L} domains such that in the fully folded Fv molecule the two residues will form a disulphide bond therebetween. Suitable methods of producing dsFv are described for example in (Glockscuther et al. Biochem. 29: 1363-1367; Reiter et al. 1994, J. Biol. Chem. 269: 18327-18331; Reiter et al. 1994, Biochem. 33: 5451-5459; Reiter et al. 1994. Cancer Res. 54: 2714-2718; Webber et al. 1995, Mol. Immunol. 32: 249-258).

Also contemplated as antigen-binding molecules are single variable region domains (termed dAbs) as for example disclosed in (Ward et al. 1989, Nature 341: 544-546; Hamers-Casterman et al. 1993, Nature. 363: 446-448; Davies & Riechmann, 1994, FEBS Lett. 339: 285-290).

Alternatively, the antigen-binding molecule may comprise a “minibody”. Minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Nail Acad. Sci. USA, 92: 652-6556) which discloses a four-helix bundle protein cytochrome b562 having two loops randomised to create complementarity determining regions (CDRs), which have been selected for antigen binding.

The antigen-binding molecule may be multivalent (ie. having more than one antigen-binding site). Such multivalent molecules may be specific for one or more antigens. Multivalent molecules of this type may be prepared by dimerisation of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerisation may be facilitated by fusion of the antibody fragments to amphiphilic helices that naturally dimerise (Pack P. Plünckthun, 1992, Biochem. 31: 1579-1584), or by use of domains (such as the leucine zippers jun and fos) that preferentially heterodimerise (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra).

The antigen-binding molecules can be used to screen expression libraries for variant aberrant polypeptides of the invention as described herein. They can also be used to detect aberrant polypeptides, aberrant polypeptide fragments, variants and derivatives of the invention as described hereinafter.

7. Methods of Detecting Aberrant Expression of KLK4 or K4

7.1 Assays for Detecting Modulation of the Level and/or Functional Activity of K4

The present invention is predicated in part on the discovery that aberrant KLK4 polynucleotide and aberrant K4 polypeptide are expressed in cancers and/or in benign tumours but not in normal tissues and that K4 and/or aberrant K4 polypeptides are expressed at a higher level in cancers than in normal tissues. Thus, the invention also features a method for detecting the presence or diagnosing the risk of at least one condition selected from a cancer or a benign tumour in a patient, comprising detecting aberrant expression of KLK4 in a biological sample obtained from said patient.

Suitably, the method comprises detecting a change in the level and/or functional activity of an expression product of a gene selected from KLK4 or a gene belonging to the same regulatory or biosynthetic pathway as KLK4, wherein the change is relative to a normal reference level and/or functional activity. In one embodiment of this type, the method comprises detecting said change in a cell of prostatic origin, wherein said cell is selected from a basal cell, a stem cell that is a precursor of, or differentiates into, an epithelial cell or a malignant cancer cell, a cell of a precursor lesion to a cancer or a cell of a prostatic intra-epithelial neoplasia (PIN). In another embodiment of this type, the method comprises detecting said change in a bone metastasis, which is preferably associated with an ovarian cancer or an endometrial cancer, and more preferably with a prostate cancer. Preferably, the method comprises detecting a reduction or abrogation in the level and/or functional activity of a KLK4 expression product relative to said corresponding normal reference level and/or functional activity. Preferably, the level and/or functional activity of said expression product in said biological sample is at least 110%, more preferably at least 200%, even more preferably at least 300%, even more preferably at least 500%, even more preferably at least 1000%, even more preferably at least 2000%, even more preferably at least 4000%, even more preferably at least 6000%, even more preferably at least 8000%, and still more preferably at least 10,000% of that which is present in a corresponding biological sample obtained from a normal individual or from an individual who is not afflicted with said condition.

Any method of directly or indirectly detecting modulation in the level and/or functional activity of the said expression product is encompassed by the present invention. For example, such detection can be achieved utilising techniques including, but not restricted to, immunoassays such as Western blotting and ELISAs, and RT-PCR. Exemplary immunoassays, which could be used for these purposes, are described for example in Section 7.2. For example, in one embodiment, a biological sample from a patient is contacted with an antigen-binding molecule that is specifically immuno-interactive with K4. The concentration of a complex comprising the polypeptide and the antigen-binding molecule is measured in the contacted sample and the measured complex concentration is then related to the concentration of the polypeptide in the sample. Preferably, the concentration of said polypeptide is compared to a reference or baseline level of said polypeptide corresponding to normal tissues. The presence of the cancer or benign tumour is diagnosed if the concentration of the polypeptide corresponds to a non-reference level concentration.

It will also be appreciated that assays may detect or measure modulation of a genetic sequence from which the target protein of interest is regulated or expressed. In another example, the subject of detection could be an upstream regulator of KLK4/K4, or a downstream regulatory target of KLK4/K4, instead of KLK4/K4.

7.2 Detection of Normal and Aberrant Molecules

It is to be understood that although the following discussion is specifically directed to human patients, the teachings are also applicable to any animal that expresses an aberrant KLK4 polynucleotide or an aberrant K4 polypeptide which correspond to a defect in KLK4 gene or transcript structure or to a defect in K4 protein structure, such that clinical manifestations particularly those seen in patients with at least one condition selected from cancer and benign tumour are found. The teachings are also applicable to any animal in which a wild-type or aberrant K4 polypeptide is present in the nucleus of a cell, and whose presence correlates with the presence or risk of a cancer or a benign tumour. It will also be appreciated that the methods described herein are applicable to any patient suspected of developing, or having, a said condition, whether such condition is manifest at a young age or at a more advanced age in a patient's life.

The diagnosis of a cancer is facilitated, or a predisposition therefor is suggested, in one embodiment, by detecting the presence of a wild-type or aberrant K4 polypeptide in the nucleus of a cell, which is preferably selected from an ovarian cell, an endometrial cell and a prostate cell. In an especially preferred embodiment of this type, the cell is a prostate or an ovarian cell. In one embodiment of this type, the aberrant K4 polypeptide comprises an insertion relative to normal K4, which preferably comprises the sequence set forth in SEQ ID NO: 9. More preferably, the aberrant K4 polypeptide comprises the sequence set forth in SEQ ID NO: 2. The cell is preferably selected from an ovarian cell, an endometrial cell and a prostate cell and more preferably selected from a prostate or an ovarian cell.

In another embodiment, diagnosis of a cancer is facilitated, or a predisposition therefor is suggested, by detecting the presence of an aberrant KLK4 polynucleotide, or an expression product thereof, in a cell such as but not limited to a basal cell, a stem cell that is a precursor of, or differentiates into, an epithelial cell or a malignant cancer cell, a cell of a precursor lesion to a cancer or a cell of a prostatic intra-epithelial neoplasia (PIN).

In yet another embodiment, diagnosis of a cancer is facilitated, or a predisposition therefor is suggested, by detecting the presence of an aberrant KLK4 polynucleotide, or an expression product thereof, in a bone metastasis.

7.2.1 Screening for Aberrant K4 Polypeptides

Detecting the presence or diagnosing the risk of a cancer or a benign tumour in a patient is now possible by detecting an aberrant K4 polypeptide that correlates with that condition. For example, the presence or absence of an aberrant K4 polypeptide with said correlation in a patient may be determined by isolating a biological sample from a patient, contacting the sample with an antigen-binding molecule as described in Section 6 and detecting the presence of a complex comprising the antigen-binding molecule and the aberrant polypeptide in the contacted sample.

Any suitable technique for determining formation of the complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to “CURRENT PROTOCOLS IN IMMUNOLOGY” (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4, 424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including serum, whole blood, and plasma or lymph fluid. The sample is, therefore, generally a circulatory sample comprising circulatory fluid.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

Two site assays are preferred for detecting truncation variants of K4 such as the aberrant K4 polypeptide set forth in SEQ ID NO: 4. In this respect, an unlabelled antigen-binding molecule immobilised on a solid substrate and that is specific for a truncation variant can be used as a capturing antibody. A second labelled antigen-binding molecule that is specific to an epitope downstream of where the truncation variant terminates can than be used to interrogate the bound antigen. The absence of any bound, labelled second antigen-binding molecule is indicative that the captured antigen is a K4 truncation variant.

An alternative assay involves immobilising the antigen in the biological sample and then exposing the imnmobilised antigen to specific antibody that may or may not be labelled with a reporter molecule. Such an assay may be suitable for detecting the aberrant K4 polypeptide set forth in SEQ ID NO: 2. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in United States Patent Specifications U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodates. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

7.3 Screening for Aberrant KLK4 Polynucleotides

In another embodiment, the invention provides a method of screening a patient for an aberrant KLK4 polynucleotide that correlates with the presence or risk of at least one condition selected from a cancer or benign tumour. The method comprises detecting said aberrant polynucleotide in a biological sample obtained from said patient using a suitable nucleic acid detection technique.

According to the invention, presymptomatic screening of a patient for the presence of a condition selected from a cancer or benign tumour or for their likelihood of developing that condition is now be possible by detecting an aberrant KLK4 polynucleotide that correlates with the presence or risk of said condition. The screening method of the invention allows a presymptomatic diagnosis, including prenatal diagnosis, for the presence of an aberrant KLK4 gene or transcript thereof in a patient and thus the basis for an opinion concerning the likelihood that that patient would develop or has developed a said condition or symptoms thereof. For example, in the method of screening, a tissue sample can be taken from a patient, and screened for the presence of one or more normal KLK4 polynucleotides. The normal human KLK4 genes can be characterised based upon, for example, detection of restriction digestion patterns in ‘normal’ versus the patient's DNA, including Restriction Fragment Length Polymorphism (RFLP) analysis, using nucleic acid probes prepared against the normal KLK4 gene(s) (or functional fragments thereof). Similarly, KLK4 mRNA may be characterised and compared to normal KLK4 mRNA levels and/or size as found in human population not at risk of developing a cancer or benign tumour using similar probes.

An aberrant KLK4 polynucleotide may be detected by determining the sequence of KLK4 genomic DNA or cDNA obtained or derived from a patient and comparing the sequence to that of wild-type KLK4 DNA or transcripts or to aberrant KLK4 transcripts described herein to thereby determine whether said sequence corresponds to an aberrant KLK4 polynucleotide. Alternatively, a nucleic acid extract from a patient may be utilised in concert with oligonucleotide primers corresponding to sense and antisense sequences of an aberrant polynucleotide sequence under test, or flanking sequences thereof, in a nucleic acid amplification reaction such as PCR, or the ligase chain reaction (LCR) as for example described in International Application WO89/09385. A variety of automated solid-phase detection techniques are also appropriate. For example, very large scale immobilised primer arrays (VLSIPS™) are used for the detection of nucleic acids as for example described by Fodor et al., (1991, Science 251:767-777) and Kazal et al., (1996, Nature Medicine 2:753-759). The above generic techniques are well known to persons skilled in the art. Preferably, at least one of said primers is an allele-specific primer specific for the aberrant polynucleotide under test. Accordingly, the present invention in another aspect contemplates a probe for interrogating nucleic acid for the presence of an aberrant KLK4 polynucleotide associated with at least one condition selected from a cancer or a benign tumour, comprising a nucleotide sequence which corresponds or is complementary to a portion of an aberrant KLK4 polynucleotide that correlates with the presence or risk of said at least one condition.

Alternatively, the presence or absence of a restriction endonuclease cleavage site resulting from a mutation or aberrant splicing in the normal KLK4 polynucleotide may be taken advantage by subjecting the aberrant polynucleotide to digestion with the restriction endonuclease. Accordingly, the present invention includes and encompasses detecting an aberrant KLK4 polynucleotide by RFLP analysis.

Alternatively, allele specific oligonucleotide primers may be used in PCR or LCR assays to detect an aberrant KLK4 polynucleotide as described above. For example, a sense primer specific for a normal KLK4 allele, a sense primer specific for an aberrant KLK4 allele may be used together with a common antisense primer. Alternatively, the two allele specific primers may be used in concert with another preferably abutting sense primer, which is complementary to a target sequence immediately adjacent and downstream of the target sequences of the allele specific primers, in LCR, in the Oligonucleotide Ligation Assay (OLA) as for example described by Landegren et al., 1988, Science 241 1077-1080.

Alternatively, the nucleic acid polymorphism in KLK4 may be detected using first-nucleotide change technology described by Dale et al. in U.S. Pat. No. 5,856,092.

The presence in the biological sample of an aberrant KLK4 size pattern, such as an aberrant KLK4 RFLP, and/or aberrant KLK4 mRNA sizes or levels and/or aberrant KLK4 polynucleotide linked to a condition described herein would indicate that the patient has developed or is at risk of developing a symptom associated with that condition.

The diagnostic and screening methods of the invention are especially useful for a patient suspected of being at risk of developing a said condition based on family history, or a patient in which it is desired to diagnose or eliminate the presence of that condition as a causative agent underlying a patient's symptoms. Prenatal diagnosis can be performed when desired, using any known method to obtain foetal cells, including amniocentesis, chorionic villous sampling (CVS), and foetoscopy.

8. Detection kits

The present invention also provides kits for the detection in a biological sample of an aberrant K4 polypeptide or aberrant polypeptide fragment, or an aberrant KLK4 polynucleotide or aberrant polynucleotide fragment. These will contain one or more particular agents described above depending upon the nature of the test method employed. In this regard, the kits may include one or more of an aberrant polypeptide or aberrant polypeptide fragment, an aberrant polynucleotide or aberrant polynucleotide fragment, a variants or derivative of these molecules a nucleic acid probe as broadly described above, or an antigen-binding molecule as broadly described above. The kits may also optionally include appropriate reagents for detection of labels, positive and negative controls, washing solutions, dilution buffers and the like. For example, a nucleic acid-based detection kit may include (i) a polynucleotide according to the invention (which may be used as a positive control), (ii) an oligonucleotide primer according to the invention, and optionally a DNA polymerase, DNA ligase etc depending on the nucleic acid amplification technique employed.

9. Identification of Target Molecule Modulators

The invention also features a method of screening for agents that can modulate the expression of a gene or the level and/or functional activity of a wild-type or aberrant expression product of said gene, wherein said gene is selected from KLK4 or a gene belonging to the same regulatory or biosynthetic pathway as KLK4. In accordance with the present invention, agents that modulate these target molecules are useful for treating and/or preventing a cancer or a benign tumour for restoring a normal level and/or functional activity of a KLK4 expression product. The screening method comprises contacting a preparation comprising at leats a portion of a wild-type or aberrant polypeptide encoded by said gene, or a variant or derivative thereof, or a genetic sequence that modulates the expression of said gene, with said agent and detecting a change in the level and/or functional activity of said polypeptide or variant or derivative, or of a product expressed from said genetic sequence.

Screening for modulatory agents according to the invention can be achieved by any suitable method. For example, the method may include contacting a cell comprising a polynucleotide corresponding to a KLK4 gene or a gene belonging to the same regulatory or biosynthetic pathway as KLK4, with an agent suspected of having said modulatory activity and screening for the modulation of the level and/or functional activity of a protein encoded by said polynucleotide, or for the modulation of the level of an expression product encoded by the polynucleotide, or for the modulation of the activity or expression of a downstream cellular target of said protein or said expression product. Detecting such modulation can be achieved utilising techniques including, but not restricted to, ELISA, cell-based ELISA, filter-binding ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a target molecule of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g. a domain such as a protein binding domain) or a portion that regulates expression of a product encoded by the polynucleotide (e.g., a promoter). For example, the promoter that is naturally associated with the polynucleotide may be introduced into the cell that is the subject of testing. In this regard, where only the promoter is utilised, detecting modulation of the promoter activity can be achieved, for example, by operably linking the promoter to a suitable reporter polynucleotide including, but not restricted to, green fluorescent protein (GFP), luciferase, B-galactosidase and catecholamine acetyl transferase (CAT). Modulation of expression may be determined by measuring the activity associated with the reporter polynucleotide.

In another example, the subject of detection could be a downstream regulatory target of the target molecule, rather than target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression and/or activity of a target molecule according to the invention.

In a series of preferred embodiments, the present invention provides assays for identifying small molecules or other compounds (i.e., modulatory agents) which are capable of inducing or inhibiting the level and/or or functional activity of target molecules according to the invention. The assays may be performed in vitro using non-transformed cells, immortalised cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, P-galactosidase or luciferase) operatively linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level and/or functional activity of the target molecule, any change in said level from an established baseline may be detected using any of the techniques described above and well known in the art. In particularly preferred embodiments, the cells are epithelial cells. Using the nucleic acid probes and/or antigen-binding molecules disclosed herein, detection of changes in the level and or functional activity of a target molecule, and thus identification of the compound as agonist or antagonist of the target molecule, requires only routine experimentation.

In particularly preferred embodiments, a recombinant assay is employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art in light of the present disclosure. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modelling, and other routine procedures employed in rational drug design.

In another embodiment, a method of identifying agents that inhibit K4 activity is provided in which a purified preparation of K4 protein in the presence and absence of a candidate agent under conditions in which K4 is active, and the level of K4 activity is measured by a suitable assay. For example, a K4 inhibitor can be identified by measuring the ability of a candidate agent to decrease K4 activity in a cell (e.g., an endometrial cell an ovarian cell or a prostate cell). In this method, a cell that is capable of expressing KLK4 is exposed to, or cultured in the presence and absence of, the candidate agent under conditions in which KLK4 is active in the cell, and an activity selected from the group consisting of tumorigenesis or benign tumour is detected. An agent tests positive if it inhibits any of these activities.

In yet another embodiment, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesised by any suitable technique. Such molecules may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 19941998), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Alternatively, a target polypeptide according to the invention may be synthesised using solution synthesis or solid phase synthesis as described, for example, in Chapter 9 of Atherton and Shephard (supra) and in Roberge et al (1995, Science 269: 202).

To identify and isolate the peptide/solid phase support that interacts and forms a complex with a target molecule, preferably a target polypeptide, it may be necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labelling with enzymes, fluorescent dyes or coloured or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a petri dish containing a substrate for either alkaline phosphatase or peroxidase, for example, S-bromo-4-chloro-3-indoyl phosphate (BCIP) or 3,3′,4,4″-diamnobenzidine (DAB), respectively. After incubating for several minutes, the peptide/solid phase-target polypeptide complex changes colour, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labelled epitope specific antigen-binding molecule. Once isolated, the identity of the peptide attached to the solid phase support may be determined by peptide sequencing.

In a preferred embodiment, the agent, which is identifiable for example by the above methods, inhibits, abrogates or otherwise reduces the expression of a gene or the level and/or functional activity of an aberrant or wild-type expression product of said gene, wherein the gene is selected from KLK4 or a gene belonging to the same regulatory or biosynthetic pathway as KLK4, for the treatment and/or prophylaxis of a cancer or benign tumour. For example, agents that may be used to reduce or abrogate gene expression include, but are not restricted to, oligoribonucleotide sequences, including anti-sense RNA and DNA molecules and ribozymes, that function to inhibit the translation, for example, of KLK4-encoding mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of an KLK4 gene, are preferred.

Ribozymes are enzymatic RNA molecules capable of catalysing the specific cleavage of RNA. The mechanism of ribozyme action involves sequence specific hybridisation of the ribozyme molecule to complementary target RNA, followed by a endonucleolytic cleavage. Within the scope of the invention are engineered hammerhead motif ribozyme molecules that specifically and efficiently catalyse endonucleolytic cleavage of KLK4 RNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridisation with complementary oligonucleotides, using ribonuclease protection assays.

Both anti-sense RNA and DNA molecules and ribozymes may be prepared by any method known in the art for the synthesis of nucleic acid molecules. These include techniques for chemically synthesising oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Various modifications to nucleic acid molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

10. Therapeutic and Prophylactic Uses

10.1 Functional K4 Polypeptides, Fragments Thereof and Modulatory Agents

A further feature of the invention is the use of a functional K4 polypeptide or biologically active fragment thereof and/or of a modulatory agent according to Section 9 as actives (“therapeutic agents”) in pharmaceutical compositions: (a) for restoring K4 function to a patient whose level and/or functional activity of normal or wild-type K4 is reduced or abrogated; (b) for restoring a normal level and/or functional activity of a KLK4 expression product; and/or (c) for treatment or prophylaxis of a condition selected from a cancer or a benign tumour.

Thus, the invention encompasses a method for restoring K4 function to a patient whose level and/or functional activity of normal or wild-type K4 is reduced or abrogated, comprising administering to the patient an effective amount of a functional KLK4 polynucleotide or biologically active fragment thereof, or a functional K4 polypeptide or biologically active fragment thereof.

The invention also extends to a method for treating or preventing the development of a condition selected from the group consisting of a cancer and a benign tumour, comprising administering to a patient in need of such treatment an effective amount of a functional KLK4 polynucleotide or biologically active fragment thereof, or a functional K4 polypeptide or biologically active fragment thereof. In a preferred embodiment, the condition is a hormone-associated condition such as a hormone-associated cancer or benign tumour. Suitably, the condition is a cancer, which is preferably selected from endometrial cancer, ovarian cancer and prostate cancer.

Also encompassed is a method for restoring a normal level of a KLK4 expression product to a patient in need of such treatment, comprising administering to said patient an effective amount of an agent as broadly described in Section 9 in the presence or absence of a pharmaceutically acceptable carrier. In a preferred embodiment, the patient has an elevated level of said expression product relative to said normal level and the administered agent reduces the level and or functional activity of said KLK4 expression product.

A pharmaceutical composition according to the invention is administered to a patient, preferably prior to such symptomatic state associated with the condition(s). The therapeutic agent present in the composition is provided for a time and in a quantity sufficient to treat that patient.

Suitably, the pharmaceutical composition comprises a pharmaceutically acceptable carrier. Depending upon the particular route of administration, a variety of pharmaceutically acceptable carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Any suitable route of administration may be employed for providing a patient with the composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier which constitutes one or more necessary ingredients. In general, the compositions are prepared by uniformly and intimately admixing the immunogenic agents of the invention with liquid carriers or finely divided solid carriers or both, and then, if necessary, shaping the product into the desired presentation.

The above compositions may be administered in a manner compatible with the dosage formulation, and in such amount as is therapeutically-effective to alleviate patients from symptoms related to the condition(s), or in amounts sufficient to protect patients from developing symptoms related to the condition(s). The dose administered to a patient, in the context of the present invention, should be sufficient to effect a beneficial response in a patient over time such as the therapeutic or prophylactic effects mentioned above. The quantity of the therapeutic agent(s) to be administered may depend on the subject to be treated inclusive of the age, sex, weight and general health condition thereof. In this regard, precise amounts of the therapeutic agent(s) for administration will depend on the judgement of the practitioner. In determining the effective amount of the therapeutic agent to be administered in the treatment of, or prophylaxis against, the condition(s), the physician may evaluate progression of the condition(s). In any event, suitable dosages of the therapeutic agents of the invention may be readily determined by those of skill in the art. Such dosages may be in the order of nanograms to milligrams of the therapeutic agents of the invention.

10.2 Functional KLK4 Polynucleotides or Fragments Thereof

A further feature of the invention is the use of a functional KLK4 polynucleotide or fragment thereof as active ingredients in a pharmaceutical composition for treating said condition(s), or preventing the development of said condition(s). Suitable functional KLK4 polynucleotides include full-length KLK4 coding sequences as for example set forth in SEQ ID NO: 5 and full-length KLK gene as for example set forth in SEQ ID NO: 12.

The functional KLK4 polynucleotides or fragments thereof are rendered expressible by operable linkage to one or more regulatory polynucleotides as for example described in Section 4 herein. This operable linkage is suitably in the form of an expression vector (another form of therapeutic agent according to the invention) which can be introduced advantageously into a patient, preferably prior to such symptomatic state associated with said condition(s). The vector is provided in a manner and an amount that permits the expression of a K4 protein provided by the functional KLK4 polynucleotide for a time and in a quantity sufficient to treat such patient.

Any suitable route of administration may be employed for providing a human or other animal the therapeutic agents of the invention as for example described in Section 10.1.

The step of introducing the expression vector into a target cell or tissue will differ depending on the intended use and species, and can involve one or more of non-viral and viral vectors, cationic liposomes, retroviruses, and adenoviruses such as, for example, described in Mulligan, R. C., (1993 Science 260: 926-932. Such methods can include, for example:

A. Local application of the expression vector by injection (Wolff et al., 1990, Science 247: 1465-1468), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of said protein.

B. General systemic delivery by injection of DNA, (Calabretta et al., 1993, Cancer Treat. Rev. 19: 169-179), or RNA, alone or in combination with liposomes (Zhu et al., 1993, Science 261: 209-212), viral capsids or nanoparticles (Bertling et al., 1991, Biotech. Appl. Biochem. 13: 390-405) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by said expression vector, or of cells responsive to said protein.

C. Injection or implantation or delivery by any means, of cells that have been modified ex vivo by transfection (for example, in the presence of calcium phosphate: Chen et al., 1987, Mole. Cell Biochem. 7: 2745-2752, or of cationic lipids and polyamines: Rose et al., 1991, BioTech. 10: 520-525), infection, injection, electroporation (Shigekawa et al., 1988, BioTech. 6: 742-751) or any other way so as to increase the expression of said polynucleotide in those cells. The modification can be mediated by plasmid, bacteriophage, cosmid, viral (such as adenoviral or retroviral; Mulligan, 1993, Science 260: 926-932; Miller, 1992, Nature 357: 455-460; Salmons et al., 1993, Hum. Gen. Ther. 4: 129-141) or other vectors, or other agents of modification such as liposomes (Zhu et al., 1993, Science 261: 209-212), viral capsids or nanoparticles (Bertling et al., 1991, Biotech. Appl. Biochem. 13 390-405), or any other mediator of modification. The use of cells as a delivery vehicle for genes or gene products has been described by Barr et al., 1991, Science 254: 1507-1512 and by Dhawan et al., 1991, Science 254 1509-1512. Treated cells can be delivered in combination with any nutrient, growth factor, matrix or other agent that will promote their survival in the treated subject.

The invention further contemplates cells or tissues containing therein a vector of the invention, or alternatively, cells or tissues produced from the treatment method of the invention.

In order that the invention may be readily understood and put into practical effect, particular preferred embodiments will now be described by way of the following non-limiting examples.

### EXAMPLES

### Example 1

Tumour Samples and Cell Culture

The endometrial cancer cell lines, Ishikawa (well differentiated), HEC1A, HEC1B, RL95-2 (moderately differentiated) and KLE (poorly differentiated) and the prostate cancer cell line LNCaP, used for a control, were obtained from the American Type Culture Collection, Rockville, Md. Prostate and kidney control tissue was obtained from Royal Brisbane Hospital and Princess Alexandra Hospital respectively. All cell lines were cultured in DMEM (Life Technologies, Rockville, Md.) with 10% FCS, 50 U/mL Penicillin G and 50 μg/mL Streptomycin (CSL Biosciences, Melbourne, Australia) at 37 C and 5% CO₂. The regulation studies were performed in triplicate in the KLE cell line using phenol red and FCS-free media. After 24 h, fresh media was supplemented with 10 nmol/L 17β-estradiol or progesterone, (Sigma Chemical Company, St Louis, Mo.) and the cells were maintained for 48 h with these steroids. In addition, 10 mmol/L progesterone was added to one group of oestrogen-treated cells after 24 h.

Normal ovary and ovarian tumour samples were obtained at surgery from women who underwent laparotomy for benign and malignant conditions in the Department of Obstetrics and Gynaecology at the Royal Women's Hospitals and Monash Medical Centre. Ethics approval was obtained from the respective institutional Ethics Committees and informed consent was obtained from all patients. Epithelial cells from normal, benign and malignant ovaries were isolated from some of these tissue samples and the primary cultured cells were grown in M199 (Sigma, St. Louis, Mo., USA) and MCDB 105 (Sigma) media supplemented with 10% FCS and 10 ng/mL human epidermal growth factor (Boehringer Mannheim, Germany) (17). The ovarian cancer cell lines, that were used in this study, were derived from late stage serous carcinomas with well (PEO14 and OAW42), moderate (SKOV-3 and OVCAR-3) or poor (JAM, CI-80-13S, PEO1 and PEO4) differentiation. SKOV-3 and OVCAR-3 were from American Type Culture Collection, and the remainder have been described previously (18, 19). These cell lines were grown in RPMI (Life Technologies, Inc., Gaithersburg. MD, USA) supplemented with 10% FCS. For the oestrogen regulation study, OVCAR-3 cells were grown to 50% confluency. Twenty-four hours before the experiments, the culture medium was replaced with phenol red-free RPMI containing 0.05% BSA, and 170-estradiol (Sigma) was added into the culture media at a final concentration of 100 nM. The cells were cultured for 8 hr, 16 hr, 24 hr and 30 hr respectively, and then harvested for protein extraction.

### Example 2

RT-PCR for Determining KLK4 Expression in Endometrial Cancer Cell Lines

Total RNA was extracted from triplicate cell preparations (10⁶ cells) using TRI-Reagent(Sigma) following the manufacturer's instructions. For complementary DNA (cDNA) synthesis, 2 μg of total RNA was reversed transcribed using Superscript II (Life Technologies). Primers, specific for KLK4 (5′-GCGGCACTGGTCATGGAAAACG-3′ sense, and 5′-CAAGGCCCTGCAAGTACCCG-3′ antisense), and β2-microglobulin (5′-TGAATTGCTATGTGTCTGGGT-3′ sense, and 5′-CCTCCATGATGCTGCTTACAT-3′ antisense) were used in a 20 μL reaction containing 100 ng/μL primers, 2.5 units of Platinum Taq (Life Technologies), 10 mmol/L deoxynucleotide triphosphates (DATP, dGTP, dCTP and dTTP), 10× buffer containing 1.5 mmol/L Mg²⁺ (Roche, Basel, Switzerland) and 1 μL of cDNA. The PCR cycling conditions were 94° C. for 5 min, followed by 35 cycles at 94° C. for 1 min, (62° C. for KLK4 and 55° C. for β2-microglobulin) for 1 min, and extension at 72° C. for 1 min. All PCR products were electrophoresed on a 2% agarose gel. Example 3

Southern Analysis and DNA Sequencing of PCR Products Obtained from Example 2

Southern analysis using gene specific probes was used to verify the KLK4 gene products. PCR products were transferred to a nylon membrane (Hybond-N+, Amersham Pharmacia Biotech, Little Chalfont, UK) at room temperature for approximately 16 h. Probes for KLK4, exon 4 (5′-CTACCGTGCTGCAGTGCGTG-3′) and exon 3 (5′-CTCCTACACCATCGGGCTGGGC-3′) were end-labelled with digoxigenin (DIG), added to 5 mL of DIG Easy-hyb (Roche), and incubated with the membrane overnight at 37° C. Following 3×20 min washes and a final wash in 0.2×SSC (20×SSC: 3 mol/L NaCl, 30 mmol/L sodium citrate) with 0.1% SDS at 37° C., the chemiluminescence substrate, CDP-Star™ (Roche), was used to record the hybridisation signal.

To further confirm product specificity, a number of PCR products were sequenced using the ABI PRISM Dye Terminator Cycle Sequencing Ready Reaction Kit (Perkin Elmer) at the DNA sequencing facility, University of Queensland, Brisbane.

### Example 4

RT-PCR, Southern Blot and DNA Sequencing Analysis of KLK4 in Ovarian Cancer

Total RNA was isolated from tumour cells or tissues using TRIzol™ reagent (Life Technologies, Inc.) following the manufacturer's instructions. Two μg of total RNA was reverse-transcribed into first-strand cDNA using Superscript II in a 20-μL reaction. PCR was performed with 1 μL of cDNA, 50 ng of KLK4 specific primers (exon 2 sense: 5′-GCGGCACTGGTCATGGAAAACG-3′ and exon 5 antisense: 5′-CAAGGCCCTGCAAGTACCCG-3′) and Platinum Taq (Life Technologies, Inc.). The cycling conditions were 94° C. for 5 min followed by 40 cycles of 94° C., 62° C. and 72° C. for 1 min each, and a final extension at 72° C. for 7 min. PCR for β2-microglobulin (sense primer: 5′-TGAATTGCTATGTGTCTGGGT-3′ and anti-sense primer: 5′-CCTCCATGATGCTGCTTACAT-3′) was used as an internal control with similar PCR conditions except for the annealing temperature (56° C.). The PCR products were electrophoresed on a 1.5% agarose gel and visualised by ethidium bromide staining. The resulting amplicons were analysed by Southern blot hybridisation using a digoxigenin (DIG) 3′ end-labelled KLK4 exon 3 oligonucleotide probe (5′-CTCCTACACCATCGGGCTGGGC-3′), in Easyhyb® solution (Roche) overnight at 37° C. Washes with 0.2×SSC (sodium chloride/sodium citrate)/0.1% SDS (sodium dodecyl sulphate) were performed at 37° C. The membrane was blocked with anti-DIG antibody, and signals were detected by CDP-star (Roche) using X-ray film. Some PCR products were also gel purified (QIAGEN Pty Ltd, Australia) and sequenced. DNA Sequences were analysed using TBLASTN.

### Example 5

In Situ Hybridisation Analysis of KLK4 Expression in Ovarian Cancer

Formalin fixed paraffin blocks from two serous ovarian tumours were sectioned (4 μm), deparaffinized, rehydrated and pretreated for in situ hybridisation as previously described (20). Hybridisation was performed with DIG-labelled cRNA probes overnight at 50° C. KLK4 probes were generated from a KLK4 RT-PCR product (526 bp) by cloning in p-GEM-T (Promega, WI, USA), and confirmed by sequencing analysis to verify KLK4 identity and orientation within p-GEM-T. Antisense and sense probes were generated using T7 and Sp6 RNA polymerase (Boehringer Mannheim) following NcoI and SalI digestion, respectively. Following hybridisation, sections were washed at 50° C. in 2×SSC, then at room temperature in 0.5×SSC. Sections were blocked in 1% (w/v) blocking reagent (Roche), then incubated (2 hr) with anti-DIG-alkaline phosphatase conjugated antibody (11500 dilution) (Roche). The signals were detected with 5-bromo-4-chloro-3-indolyl phosphate/nitroblue tetrazolium (BCIP/NBT) (Roche) and counterstained with fast nuclear red.

### Example 6

Western Analysis of K4 Levels in Endometrial Cancer

At 80% confluency, cells were pelleted, then lysed in ice-cold lysis buffer (Tris-pH, 7.5, 10 mmol/L; 150 mmol/L NaCl; 1% triton-X 100) containing a general protease inhibitor cocktail (Roche) and centrifuged. Total cell protein (200 μg) was boiled for 5 min and then electrophoresed on a 10% SDS-PAGE gel. The protein was then transferred to a nitrocellulose membrane (Protran™, Schleicher and Schuell, Germany) using 3-[cyclohexylamino]-1-propanesulfonic acid (CAPS) buffer (Sigma). The membrane was blocked with 5% skim milk in TBS/Tween-20 overnight. Primary anti-peptide K4 antibody (#673305, anti-C-terminal antibody), raised in this laboratory, and secondary antibody (anti-rabbit HRP-conjugate, Roche) was diluted 1:100 and 1:1000 respectively and incubated with the membrane at room temperature for 1 h. Following secondary antibody incubation and washing, Lumi-Light™ plus Western Blotting Substrate Solution (Roche) was applied directly to the membrane. Detection was determined using X-ray film and visualisation of the chemiluminescence signal. Fold changes in the level of signal were assessed using the Hewlett Packard Scan Jet IICX and ImagQuant™ 4.21A software (Molecular Dynamics, Amersham)

### Example 7

Western Analysis of K4 Levels in Ovarian Cancer

Cytoplasmic extracts (150 μg protein) from the cultured tumour cells were electrophoresed on 12% SDS-polyacrylamide gels. The protein was then transferred to a Protran membrane (Schleicher and Schuell), blocked with 5% skim milk in TBS/Tween-20 overnight at 4° C. and incubated with an affinity-purified anti-C-terminal peptide hK4 antibody (1/500 dilution, 2 hr) at room temperature. The blot was washed and incubated (1 hr) with a horseradish peroxidase goat anti-rabbit IgG (Dako, Glostrup, Denmark) (1/2,000 dilution) at room temperature and then incubated with chemiluminescent substrate and exposed to X-ray film for visualisation.

### Example 8

Immunohistochemistry Analysis of K4 Levels in Ovarian Cancer

Immunohistochemical staining was performed on the same tissue blocks as above using a Zymed Kit (Zymed Laboratories, Inc., CA, USA). Paraffin sections (4 μm) were deparaffinized and antigen retrieval was performed by microwave heat treatment in 10 mM sodium citrate buffer (pH 6.0). Following H₂O₂ treatment and blocking, the sections were incubated (2 hr) with affinity-purified anti-N-terminal and mid-region peptide hK4 antibodies (#673301, #673303) (1/250 dilution) at room temperature, then biotinylated goat anti-rabbit immunoglobulins and streptavidin-horseradish peroxidase conjugate following the manufacturer's instruction. Peroxidase activity was detected using 3,3′-aminobenzidine (DAB) (Sigma) as the chromogen with H₂O₂ as the substrate. The sections were counterstained with Mayer haematoxylin. Negative controls were performed by using normal rabbit serum instead of the primary antibody.

### Example 9

KLK4 Expression and K4 Levels in Endometrial Cancer Cell Lines

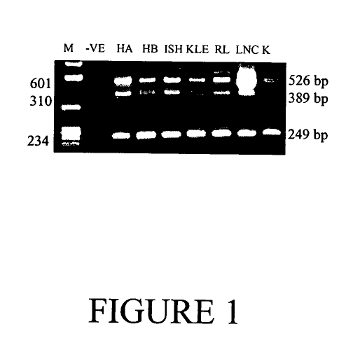

In order to confirm the RNA integrity, RT-PCR of the general “house-keeping” gene, β2-microglobulin, was used. All PCR products were of the correct size (249 bp) and thus, free of contaminating DNA (FIG. 1). The expression of KLK4 was detected in all endometrial cancer cell lines to varying degrees (FIG. 1). The positive control LNCaP and kidney control (to a lesser degree) cDNA confirmed the amplification of a product of the expected size of 526 bp. A lower band, of 389 bp was also observed in all cell lines.

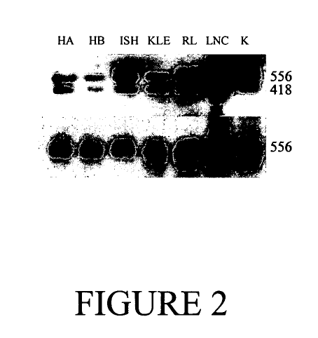

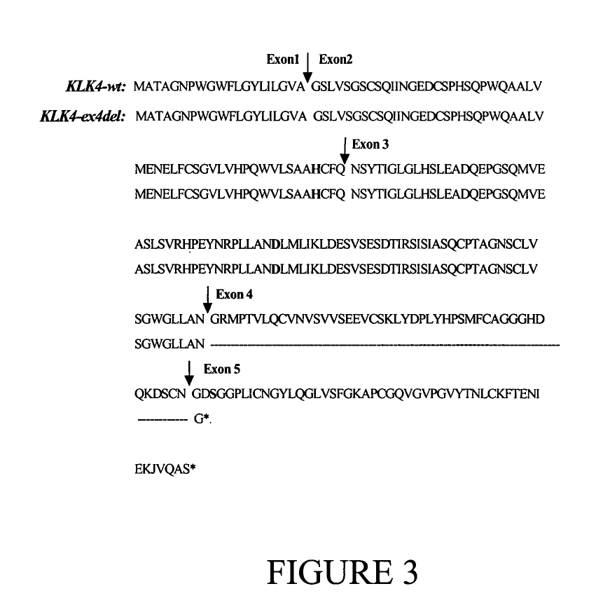

As the KLKs are a family of serine proteases with high sequence homology, we performed Southern blots on the RT-PCR products to confirm KLK4 specificity. Hybridisation with an exon 3 KLK4 probe (FIG. 2, upper panel) confirmed the expression pattern observed on the ethidium bromide stained gel. The lower 389 bp KLK4 PCR product was sequenced and shown to be a complete exon 4 deletion (FIG. 3). Subsequent analysis with an exon 4 KLK4 probe gave a single 526 bp band indicating that the lower band was indeed an exon 4 deletion product.

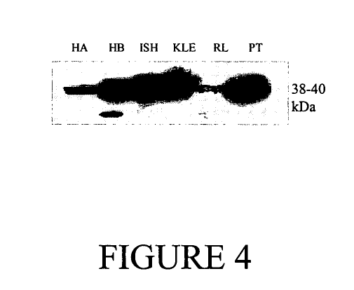

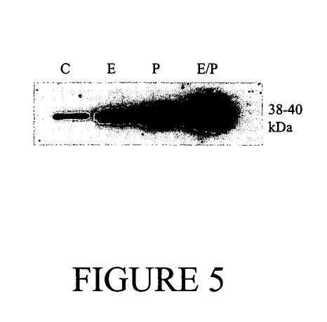

Western blotting was performed to determine the relative abundance of K4 in these cell lines. Using a K4 specific anti-C-terminal peptide antibody, we detected a protein (approx. 38-40 kDa) in all five cell lines and prostate tissue (FIG. 4). The levels of K4 protein (compared to the control) were elevated by 16, 27 and 40 fold, respectively, on the addition of 10 nmol/L estradiol, progesterone, and the combination of both, in the KLE cell line over 48 h (FIG. 5).

### Example 10

Expression of KLK4 in Normal Ovaries and Ovarian Tumours

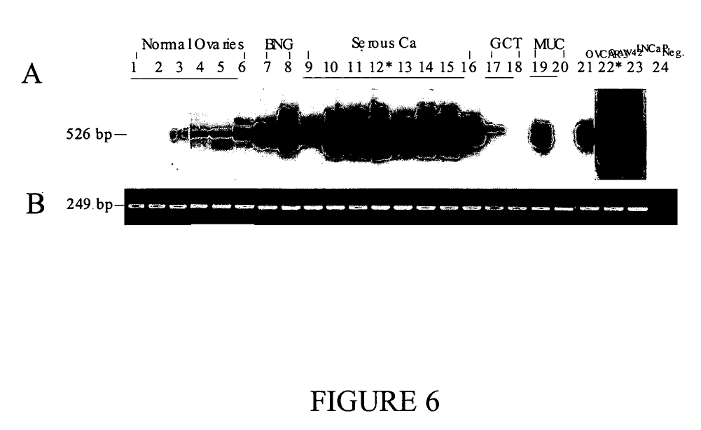

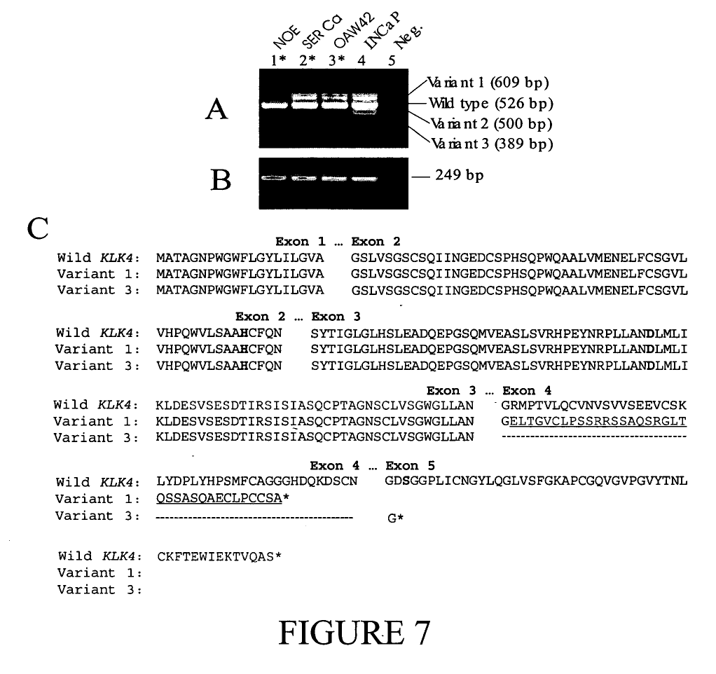

The RT-PCR Southern blot analysis of KLK4 expression in representative samples is shown in FIG. 6, with the KLK4 expression pattern and clinical information of all tumour tissues and cell lines summarised in Table 1. KLK4 expression was detected in the normal ovaries (4/6), as well as epithelial-derived serous (benign—2/2, malignant—11/11, cell lines 8/8), mucinous (benign—1/1, malignant—2/3), endometrioid (1/2) and clear cell tumours (1/1) and granulosa cell tumours (3/6) (Table 1). The level of amplified KLK4 PCR product appeared higher in the serous carcinomas than normal ovaries, mucinous and granulosa cell tumours that are KLK4 positive (FIG. 6). Indeed, a KLK4 PCR product was not observed for many of these latter tumour types and normal ovaries. In addition, eight of the nine stage III and stage 1V serous carcinomas exhibited the strongest KLK4 hybridisation signal. β2-microglobulin, which was used as an internal control (FIGS. 6B and 7B), showed a consistent pattern of expression in all samples indicating the integrity of the RNA. These results suggest that KLK4 is highly expressed by serous epithelial ovarian carcinomas.

In addition to the expected wild-type KLK4 mRNA amplicon (526 bp), three alternate splicing forms of KLK4 were observed in the ovarian cancer lines and ovarian tumour cells as well as the LNCaP control, but not in normal ovaries (FIGS. 6A and 7A). These three KLK4 variants were noted in all serous epithelial ovarian tumours, including serous adenomas (2/2), serous carcinomas (11/11), and serous carcinoma cell lines (8/8), while only 3 of 4 mucinous tumours and only 1 of 6 granulosa cell tumours showed expression of these variants (FIG. 6 and Table 1). To confirm the sequence of the KLK4 PCR products and examine the three alternate forms of KLK4, PCR products from normal ovarian epithelial cells (NOE), the primary cultured cells from a serous ovarian carcinoma and from the ovarian cancer cell line OAW42 (FIG. 7A) were sequenced. All three of these alternate sequences (variant 1, variant 2 and variant 3 in FIG. 7A). Variant 1 (FIG. 7A, 609 bp) has an 83 bp insertion of intronic sequence from intron 3, which causes a frame shift of the coding region that will generate a premature stop codon giving rise to a truncated protein product that does not contain the serine residue (Ser²⁰⁷) of the catalytic triad (FIG. 7C). Variant 2 (FIG. 7A, 400 bp) corresponds to a splice variant with an insertion of 12 bp from intron 2 as well as exon 4 deletion (FIG. 7C). Another alternate form (Variant 3 in FIG. 7A, 389 bp) has the region corresponding to exon 4 deleted (FIG. 7C) and the sequence is similar to that described in Example 9. These results were confirmed by RT-PCR using DNAse I treated RNA, indicating that this finding is not due to genomic DNA contamination.

In addition to high expression of KLK4 in ovarian carcinomas, we also observed that three KLK4 variants were detected in different ovarian tumours but not in normal ovaries. Two of these variants (variant 1, 609 bp and variant 3, 389 bp) had premature stop codons that would lead to a truncated hK4 protein, if translated. Both of these variants would not contain the Ser²⁰⁷ of the catalytic triad, and therefore they are unlikely to have enzymic activity. Previous studies from our laboratory have also reported the identification of the KLK4 variant 3 mRNA splice form in endometrial carcinoma cell lines⁵. Moreover, mRNA variants have been demonstrated for other KLKs, such as KLK1 (21), KLK2 (2), KLK3 (22, 23) and KLK13 (KLK-L4) (24). Thus, variant mRNA transcripts are a common feature of the human KLK family. Whether these variants encode functional protein remains to be clarified. Overall, we have shown increased expression of the wild-type KLK4 transcript in late stage ovarian tumours and that several KLK4 mRNA variants are expressed by ovarian tumours but not by normal ovaries. It will be important to now determine if, like PSA in prostate and breast cancer (2, 25), KLK4/hK4, or the KLK4 variant forms, could be a useful diagnostic or prognostic marker for some ovarian cancers, or monitoring this disease.

In summary, the above results show that KLK4 is differentially expressed in ovarian tumours compared with normal ovaries, with high expression of KLK4 in serous carcinomas, especially in late stage disease. KLK4 appeared to be less expressed in ovarian tumours of mucinous and granulosa cell origin. Of interest, several variant mRNA KLK4 transcripts were detected in ovarian tumours but not in normal ovaries. The above results also show that expression of KLK4 and its variants may be related to the histology and/or stage of ovarian tumours. All of the stage III and stage 1V serous ovarian carcinomas showed high levels of KLK4 expression, whilst none of the mucinous or granulosa cell tumours showed high KLK4 expression, although all of the mucinous tumours and 5 of 6 granulosa cell tumours, used in this study, were early stage tumours. All of the ovarian cancer cell lines, also showed high KLK4 expression and were epithelial-derived and from late stage serous carcinomas. These cell lines covered a spectrum from well to poorly differentiated, but no correlation between KLK4 expression and differentiation state could be drawn from this study. However, mucinous ovarian tumours and granulosa cell tumours have relatively reduced proliferative rates, when compared to serous tumours, and therefore the expression of KLK4 may be related to the proliferative status of a tumour. In addition, although all late stage ovarian cancers have poor outcomes, the prognosis of early stage serous and clear cell carcinomas is also worse than mucinous, endometrioid and granulose cell tumours. In this context, it is of interest to note, that one stage III serous carcinoma (Number 16, Table 1), a stage II endometrioid carcinoma (Number 32, Table 1) and a stage I clear cell carcinoma (Number 34, Table 1) had a better survival than the other tumours and these tumours did not show high KLK4 expression.

### Example 11

Expression of KLK4 Transcripts and hK4 in Ovarian Cancer Tissues

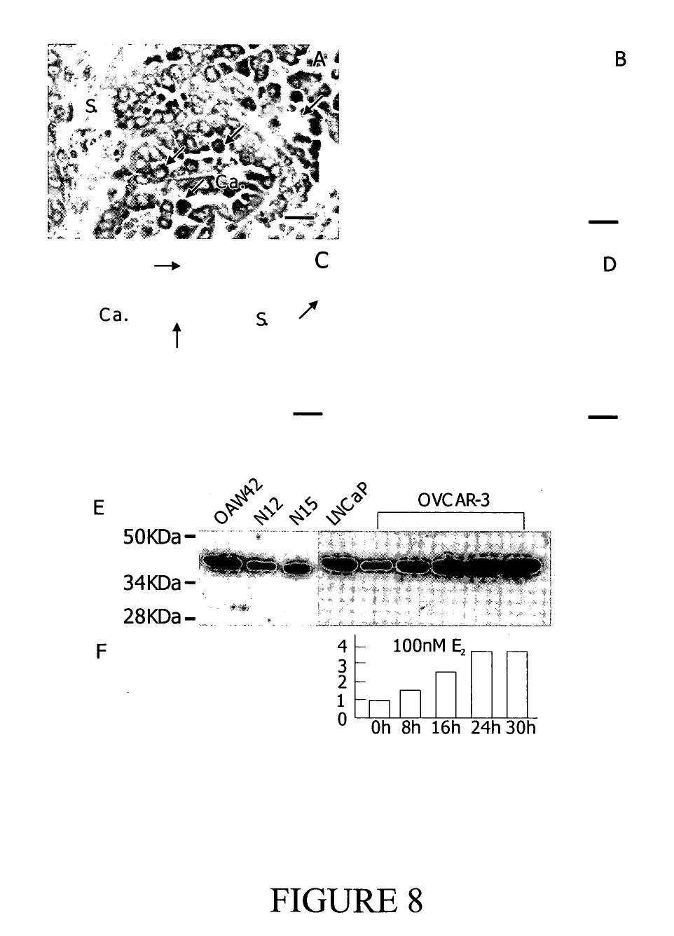

On in situ hybridisation with a DIG-labelled KLK4 antisense cRNA probe, KLK4 expression was detected in the ovarian adenocarcinoma cells of a well-differentiated serous carcinomas (FIG. 8A). Ovarian carcinoma sections hybridised with a KLK4 sense cRNA probe were negative (FIG. 8B). On immunohistochemistry, using the affinity-purified hK4 anti-N-terminal and mid-region peptide antibodies, hK4 staining was found in the cytoplasm and the membrane of ovarian carcinoma cells (FIG. 8C), while no staining was seen in the negative control (FIG. 8D).

The above results revealed that KLK4 mRNA and the hK4 protein are detected in the cytoplasm and/or cell membrane of a serous epithelial-derived adenocarcinoma cells of the tumour tissues. Consistent with the immunohistochemical staining results, the cell lysates from the ovarian carcinoma cell lines and carcinoma cells showed immunoreactivity to the hK4 antibody. The difference between the Western determined molecular weight (≈Mᵣ 40,000) and predicted (≈Mᵣ 30,000) molecular weight is probably due to a post-translation modification, as the predicted hK4 amino acid sequence contains N-glycosylation sites. The cell membrane staining was a surprising finding, as other kallikreins, such as PSA, are secreted enzymes and usually localised to the cytoplasm. However, there are several predicted myristylation sites in the hK4 sequence that may indicate a cell membrane function.

### Example 12

Western Blot Analysis and Oestrogen Regulation of hK4

The affinity-purified anti-C-terminal peptide hK4 antibody recognises a protein of ≈Mᵣ 40,000 in the prostate cancer cell line LNCaP, ovarian cancer cell line OAW42 and primary cultured serous ovarian carcinoma cells N12 and N15 (FIG. 8E). Similar results were obtained with other affinity-purified antibodies to peptides from different regions of the putative hK4 protein (data not shown). The oestrogen receptor-positive ovarian carcinoma cell line OVCAR-3 was used to evaluate whether hK4 expression is under oestrogen regulation. As shown in FIG. 8E, a 1.5-4 fold up-regulation of hK4 intracellular levels by 100 nM oestrogen was found and this regulation was time dependent (FIG. 8F).

### Example 13

Cellular Localisation of K4 in the Normal Ovarian Epithelial and Ovarian Cancer Cells

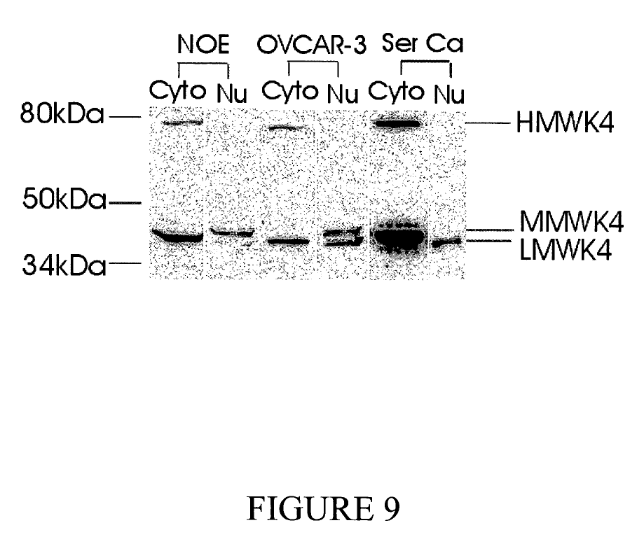

Using a C-terminal directed antibody and Western blot analysis, the present inventors have/determined that K4 protein is localised both in the nuclei and cytoplasm of normal ovarian epithelial (NOE) and OVCAR-3 cancer cells (FIG. 9). This is very novel as the kallikreins are considered secreted proteins and are typically localised in the cytoplasm but this finding suggests a nuclear role as well. Although no conventional nuclear localisation motif (NLS) motif is present in K4, using the PSORT protein prediction model (http://psort.nibb.acjp), a C terminal fragment of K4 that includes the peptide against which we have raised this antibody is predicted to be nuclear localised. Moreover, the predicted protein of the variant 1 K4 transcript (see FIG. 7C) is also predicted to be nuclear localised. Of interest, it has been recently shown that the normally membrane-bound enzyme, ADAM10, can also be localised to the nucleus in prostate cancer cells (Akl & Herington—personal communication). Furthermore, a known target protein of the kallikrein K3 (PSA), IGFBP-3, can be transported to the nucleus (26), thus it follows that the (co-) nuclear transport of its enzymic interacting protein may occur under some circumstances. Inspection of FIG. 9 also reveals that the nuclear pattern (expected 40 kDa size+a 45 kDa band; LMWK4 and MMWK4 of FIG. 9) is different from that seen in the cytoplasm (expected 40 kDa size+80 KDa dimer or K4/protein complex; LMWK4 and HMWK4 of FIG. 9). Although several known binding proteins or inhibitors bound to the kallikreins are larger than 40 kDa (27), the kallistatin and PI-6 inhibitors are of a comparable size (40 kDa and 30 kDa) and are known to bind K1 and prostatic cytosolic K2 respectively (28, 29). Consistent with these data of elevated KLK4 expression in serous derived tumours and cancer cell lines (FIG. 6), the levels of K4, both cytoplasmic and nuclear, are elevated in the cancer cell lines (FIG. 9). Further inspection of FIG. 9 reveals the presence of different molecular weight K4-containing species between NOE and serous derived tumours and cancer cell lines. In this regard, the nuclear extract of NOE primarily contained the 45 kDa band (MMWK4). In contrast, the nuclear extract of OVCAR-3 contained approximately equal amounts of the 40 kDa and 45 kDa bands (LMWK4, MMWK4) whereas the nuclear extract of SER Ca principally contained the 40 kDa band (LMWK4).

### Example 14

Cellular Localisation of KLK4 and K4 in the Prostate and its Association With Cancer Progression

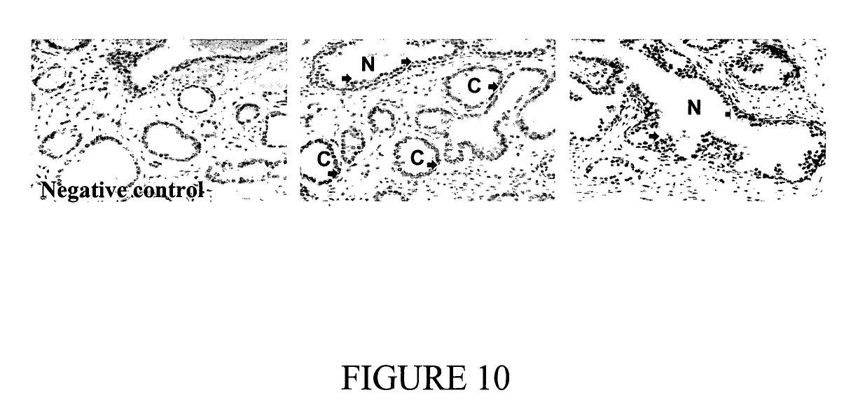

Using in situ hybridisation with a digoxigenin (DIG)-labelled cRNA probe for KLK4, the presence of KLK4 mRNA was detected in the secretory cells of the prostate gland in both normal, hyperplastic and adenocarcinoma tissues (data not shown). The control sense hybridisation was appropriately negative. A further control pre-hybridisation with cold antisense RNA for KLK4 and KLK3 (PSA) confirmed the specificity of the hybridisation. Immunohistochemistry was performed with specific K4 polyclonal antibodies raised against three synthetic peptides derived from different regions of the K4 protein (an N-terminal portion, residues 31 through 41; a central portion, residues 101 through 113; and a carboxy terminal portion, residues 174 through 189). The K4 protein was localised in the cytoplasm and the nucleus of the secretory epithelial cells of the prostate gland. Pre-absorption with K4 purified peptides was performed to determine the specificity of the staining. The intensity of K4 staining (cytoplasm and nucleus) appeared lowest in normal glands and increased with cancer progression, appearing highest in later stage adenocarcinoma (see FIG. 10). In summary, these data show that KLK4/K4 is expressed in the cytoplasm and nucleus of epithelial cells of normal/benign prostate and that increased levels of K4 are highly associated with advanced disease.

### Example 15

Immunohistochemical Staining of K4 in PIN

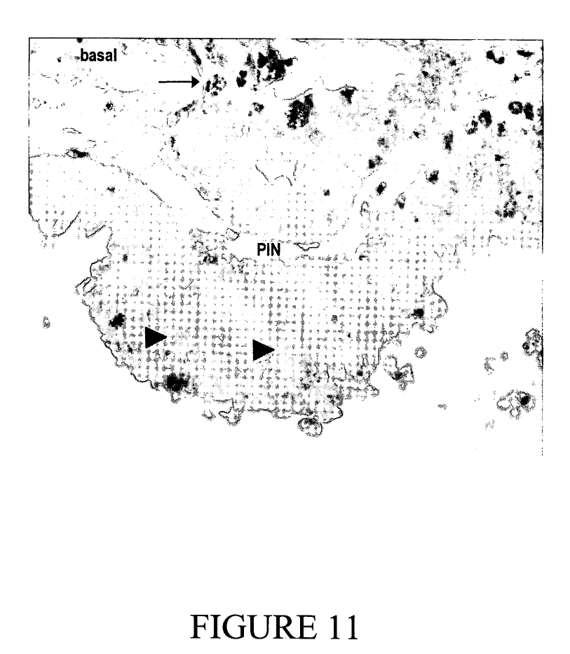

Immunohistochemical staining was performed with an anti N-terminal peptide K4 antibody on sections containing high grade PIN lesions from men with prostate cancer. The most intense and extensive staining was detected in the PIN lesions (FIG. 11) as well as cancer (data not shown). In PIN lesions, both secretory and basal cells gave strong cytoplasmic staining. K4 staining was also found frequently in the nucleus of the secretory cells of high grade PIN (big arrow) but not in the nucleus of the basal cells (small arrow). These findings indicate that with two different antibodies to K4 (directed against the N terminus or C terminus—FIGS. 10 and 11), nuclear staining can be detected, although the N terminal antibody detects predominantly cytoplasmic staining in normal or benign glands (data not shown).

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

Tables

TABLE 1
Patient characteristics and expression patterns of KLK4.

| | | | | KLK4 | Summary of KLK4 |
|---|---|---|---|---|---|
| | | | Survival | expression | positive tumors in |
| Number | Histology | Stage⁸/Grade⁹ | Months | (Variants)¹⁰ | different histology |
| 1 | NOE¹ | | | 1+ (−) | |
| 2 | NOE | | | 1+ (−) | |
| 3 | NOE | | | 1+ (−) | |
| 4 | NOT² | | | — | |
| 5 | NOT | | | — | |
| 6 | NOT | | | 1+ (−) | 4/6 |
| 7 | SER³ adenoma | | | 1+ (+) | |
| 8 | SER adenoma | | | 2+ (+) | 2/2 |
| 9 | SER Ca | IIb/2 | 20 | 1+ (+) | |
| 10 | SER Ca | IIc/3 | 2 | 2+ (+) | |
| 11 | SER Ca | IIIc/1 | 66 | 2+ (+) | |
| 12 | SER Ca | IV/2-3 | 22 | 2+ (+) | |
| 13 | SER Ca | IIIc/3 | 25 | 2+ (+) | |
| 14 | SER Ca | IIIc/3 | 19 | 2+ (+) | |
| 15 | SER Ca | IIIb/3 | 18 | 2+ (+) | |
| 16 | SER Ca | III/1 | 162 | 1+ (+) | |
| 17 | SER Ca Tissue | III/3 | | 2+ (+) | |
| 18 | SER Ca Tissue | III/2-3 | | 2+ (+) | |
| 19 | SER Ca Tissue | III/3 | | 2+ (+) | 11/11 |
| 20 | JAM (SER) | Xenograft/3 | | 2+ (+) | |
| 21 | CI-80-13S (SER) | IV/3 | | 2+ (+) | |
| 22 | SKOV-3 (SER) | III/1 | | 1+ (+) | |
| 23 | OVCAR-3 (SER) | III/NA¹¹ | | 3+ (+) | |
| 24 | PEO1 (SER) | III/3 | | 2+ (+) | |
| 25 | PEO4 (SER) | Recurrent | | 1+ (+) | |
| 26 | PEO14 (SER) | III/1 | | 3+ (+) | |
| 27 | OAW42 (SER) | III/NA | | 3+ (+) | 8/8 |
| 28 | MUC⁴ adenoma | | | 1+ (+) | |
| 29 | MUC Ca | I | | — | |
| 30 | MUC Ca | I | | 1+ (+) | |
| 31 | MUC Ca | II | | 1+ (−) | ¾ |
| 32 | END⁵ Ca | IIb/2 | 84 | — | |
| 33 | END Ca | III/2-3 | 10 | 2+ (+) | 1/2 |
| 34 | CCC⁶ | Ia/2 | 124 | 1+ (+) | 1/1 |
| 35 | GCT⁷ | I/NA | | 1+ (+) | |
| 36 | GCT | I/NA | | — | |
| 37 | GCT | I/NA | | 1+ (−) | |
| 38 | GCT | Ia/NA | | — | |
| 39 | GCT | Unstaged/NA | | — | |
| 40 | GCT | Recurrent/NA | | 1+ (−) | 2/6 |

¹NOE, normal ovarian epithelial cells;
²NOT, normal ovarian tissues;
³SER, serous;
⁴MUC, mucinous;
⁵END, endometrioid;
⁶CCC, clear cell carcinoma;
⁷GCT, granulosa cell tumour;
⁸Federation of International Gynaecology and Obstetrics (FIGO) stage system.
⁹Serov and Scully, 1973, 1, 2, 3 and 4: well, moderately, poorly and undifferentiated;
¹⁰Intensity of KLK4 RT-PCR bands:
—, no expression,
1+ to 3+ indicates increasing intensity of the RT-PCR products;
Presence (+) or absence (−) of KLK4 mRNA variants.
¹¹NA, not available.

### BIBLIOGRAPHY

Clements J A, 1989. The glandular kallikrein family of enzymes: tissue-specific expression and hormonal regulation. Endocr Rev. 10: 393-419.

2. Rittenhouse, H. G., Finlay J. A., Mikolajczyk S. D. and Partin A. W. 1998. Human kallikrein 2 (hK2) and prostate-specific antigen (PSA); two closely related, but distinct, kallikreins in the prostate. Crit. Rev. Clin. Lab. Sci., 35: 275-386.

3. Magklara A, Grass L and Diamandis EP. 2000. Differential steroid hormone regulation of human glandular kallikrein (hK2) and prostate-specific antigen (PSA) in breast cancer cell lines. Breast Cancer Res Treat. 59:263-270.

4. Clements J and Mukhtar A, 1994. Glandular kallikreins and prostate-specific antigen are expressed in the human endometrium. J Clin Endocrinol Metab. 78: 1536-1539.

5. Corthom J and Valdes G. 1994. Variations in uterine kallikrein during cycle and early pregnancy in the rat. Biol Reprod. 50: 1261-1264.

6. Clements J and Mukhtar A, 1997. Tissue kallikrein and the bradykinin B2 receptor are expressed in endometrial and prostate cancers. Immunopharmacology. 36: 217-220.

7. Anisowicz, A., Sotiropoulou, G., Stenman, G., Mok, S. C., and Sager, R. 1996. A novel protease homolog differentially expressed in breast and ovarian cancer. Mol Med., 2: 624-636.

8. Tanimoto, H., Underwood, L. J., Shigemasa, K., Yan Yan, M. S., Clarke, J., Parmley, T. H., and O'Brien, T. J. 1999. The stratum corneum chymotryptic enzyme that mediates shedding and desquamation of skin cells is highly overexpressed in ovarian tumor cells. Cancer, 86: 2074-2082.

9. Underwood, L. J., Tanimoto, H., Wang, Y., Shigemasa, K., Parmley, T. H., and O'Brien, T. J. 1999. Cloning of tumor-associated differentially expressed gene-14, a novel serine protease overexpressed by ovarian carcinoma. Cancer Res., 59: 4435-4439.

10. Nelson, P. S., Gan, L., Ferguson, C., Moss, P., Gelinas, R., Hood, L., and Wang, K. 1999. Molecular cloning and characterization of prostase, an androgen-regulated serine protease with prostate-restricted expression. Proc Natl Acad Sci USA, 96: 3114-3119.

11. Yousef, G. M., Obiezu, C. V., Luo, L. Y., Black, M. H., and Dianandis, E. P. 1999. Prostase/KLK-L1 is a new member of the human kallikrein gene family, is expressed in prostate and breast tissues, and is hormonally regulated. Cancer Res., 59: 4252-4256.12.

12. Stephenson, S. A., Verity, K., Ashworth, L. K., and Clements, J. A. 1999. Localization of a new prostate-specific antigen-related serine protease gene, KLK4, is evidence for an expanded human kallikrein gene family cluster on chromosome 19q13.3-13.4. J Biol Chem., 274: 23210-23214.

13. Hu, J. C., Zhang, C., Sun, X., Yang, Y., Cao, X., Ryu, O. and Simmer, J. P. 2000. Characterization of the mouse and human PRSS17 genes, the relationship to other serine proteases, and the expression of PRSS17 in developing mouse incisors. Gene, 251: 1-8.

14. Overall C M and Limeback H. 1988. Identification and characterization of enamel proteinases isolated from developing enamel. Biochem J. 256: 965-972.

15. Simmer, J. P., Fukae, M., Tanabe, T., Yamakoshi, Y., Uchida, T., Xue, J., Margolis, H. C., Shimizu, M., DeHart, B. C., Hu, C. C., and Bartlett, J. D. 1998. Purification, characterization, and cloning of enamel matrix serine proteinase 1. J Dent Res., 77: 377-386.

16. Hansson L, Stromvist M, Backman A, Wallbrandt P, Calstein A and Egelrud T. 1994. Cloning, expression, and characterisation of stratum corneum chymotryptic enzyme. J. Biol. Chem. 269: 19420-19426.

17. Godwin, A. K., Testa, J. R., Handel, L. M., Liu, Z., Vanderveer, L. A., Tracey, P. A. and Hamilton, T. C. 1992. Spontaneous transformation of rat ovarian surface epithelial cells: association with cytogenetic changes and implications of repeated ovulation in the etiology of ovarian cancer. J Natl Cancer Inst., 84: 592-601.

18. Clark, S., McGuckin, M. A., Hurst, T., and Ward, B. G. 1994. Effect of interferon gamma and tumour necrosis factor alpha on sensitivity to cisplatin in ovarian carcinoma cell lines. Cancer Immunol Immunother., 39: 100-104.

19. Dong, Y., Berners-Price, S. J., Thorbum, D. R., Antalis, T., Dickinson, J., Hurst, T., Qiu, L., Khoo, S. K., and Parsons, P. G. 1997. Serine protease inhibition and mitochondrial dysfunction associated with cisplatin resistance in human tumor cell lines: targets for therapy. Biochem Pharmacol., 53: 1673-1682.

20. Clements, J. A., Mukhtar, A., Holland, A. M., Ehrlich, A. R., and Fuller, P. J. 1995. Kallikrein gene family expression in the rat ovary: localization to the granulosa cell. Endocrinology, 136: 1137-1144.

21. Rae, F., Bulmer, B., Nicol, D., and Clements, J. 1999. The human tissue kallikreins (KLKs 1-3) and a novel KLK1 mRNA transcript are expressed in a renal cell carcinoma cDNA library. Immunopharmacology, 45: 83-88.

22. Heuze, N., Olayat, S., Gutman, N., Zani, M. L., and Courty, Y. 1999. Molecular cloning and expression of an alternative hKLK3 transcript coding for a variant protein of prostate-specific antigen. Cancer Res., 59: 2820-2824.

23. Tanaka, T., Isono, T., Yoshiki, T., Yuasa, T., and Okada, Y. 2000. A novel form of prostate-specific antigen transcript produced by alternative splicing. Cancer Res., 60: 56-59.

24. Yousef, G., Chang, A., and Diamandis, E., P. 2000. Identification and characterization of KLK-L4, a new kallikrein-like gene that appears to be down-regulated in breast cancer tissues. J Biol Chem., 275: 11891-11898.

25. Webber, M. M., Waghray, A., and Bello, D. 1995. Prostate-specific antigen, a serine protease, facilitates human prostate cancer cell invasion. Clin Cancer Res., 1: 1089-1094.

26. Schedlich L J, Young T F, Firth S M, Baxter R C. 1998. Insulin-like growth factor-binding protein (IGFBP)-3 and IGFBP-5 share a common nuclear transport pathway in T47D human breast carcinoma cells. J Biol Chem 273(29): 18347-18352.

27. Rittenhouse H G, Finlay J A, Mikolajczyk S D, Partin A W. 1998. Human Kallikrein 2 (hK2) and prostate-specific antigen (PSA): two closely related, but distinct, kallikreins in the prostate. Crit Rev Clin Lab Sci. 35(4): 275-368.

28. Chen V C, Chao L, Chao J. 2000. A positively charged loop on the surface of kallistatin functions to enhance tissue kallikrein inhibition by acting as a secondary binding site for kallikrein. J Biol Chem 275(51): 40371-40377.

29. Mikolajczyk S D, Millar L S, Marker K M, Rittenhouse H G, Wolfert R L, Marks L S, Charlesworth M C, Tindall D J. 1999. Identification of a novel complex between human kallikrein 2 and protease inhibitor-6 in prostate cancer tissue. Cancer Res. 59(16): 3927-3930.

1. A method for detecting the presence or diagnosing the risk of a cancer or benign tumour associated with an organ or tissue selected from the group consisting of ovaries, endometrium and prostate in a patient, comprising detecting in a basal cell from said patient a change in the level and/or functional activity of an expression product of a gen selected from KLK4 or a gene belonging to the same regulatory or biosynthetic pathway as KLK4, wherein the change is relative to a normal reference level and/or functional activity.

2. The method of claim 1, wherein said change is detected in a basal cell of prostatic origin.

3. The method of claim 1, wherein said change is detected in a stem cell.

4. The method of claim 1, wherein said change is detected in a stem cell of prostatic origin, which is a precursor of, or differentiates into, an epithelial cell or a malignant cancer cell.

5. The method of claim 1, who said change is detected in a precursor lesion to a cancer.

6. The method of claim 1, wherein said change is detected in a prostatic intra-epithelial neoplasia (PIN).

7. The method of claim 1, wherein said change is detected in a bone metastasis.

8. The method of claim 1, wherein said change is detected in a bone metastasis associated with a cancer selected from an ovarian cancer, an endometrial cancer or a prostate cancer.

9. The method of claim 1, wherein said change is detected in a bone metastasis associated with a prostate cancer.

10. The method of claim 1, wherein said change is detected in the nucleus of a cell.

11. The method of claim 1, wherein said change is detected in the nucleus of a cell selected from an endometrial cell a prostate cell or an ovarian cell.

12. The method of claim 1, wherein said change is detected in the nucleus of a cell selected from a prostate cell or an ovarian cell.

13. The method of claim 1, wherein the level and/or functional activity of said expression product in said biological sample is at least 10% of that which is present in corresponding biological sample obtained from a normal individual or from an individual who is not afflicted with said condition.

14. A method for detecting the presence or diagnosing the risk of a cancer or benign tumour associated with an organ or tissue selected from the group Consisting of ovaries, endometrium and prostate in a patient, comprising determining the presence of an aberrant KLK4 expression product in a biological sample obtained from said patient, wherein said aberrant expression product is selected from an aberrant K4 polypeptide with impaired, altered or abrogated function relative to normal K4, wherein said aberrant K4 polypeptide comprises the sequence set forth in any one of SEQ ID NO: 2, 4 and 15, or an aberrant K4 polynucleotide encoding said aberrant K4 polypeptide.

15. The method of claim 14, wherein the presence of said aberrant K4 polypeptide is detected in to nucleus of a cell.

16. The method of claim 14, wherein the presence of said aberrant K4 polypeptide is detected in the nucleus of a cell selected from an endometrial cell, a prostate cell or an ovarian cell.

17. The method of claim 14, wherein the presence of said aberrant K4 polypeptide is detected in the nucleus of a cell selected form a prostate cell or an ovarian cell.

18. The method of claim 14, wherein the aberrant K4 polypeptide has a molecular weight that is lower than the molecular weight of a K4 polypeptide present in the nucleus of a normal cell.

19. The method of claim 14, wherein the aberrant K4 polypeptide comprises an insertion relative to normal K4, comprising the sequence set forth in SEQ ID NO: 9.

20. The method of claim 14, wherein the aberrant K4 polypeptide comprises the sequence set fort in SEQ ID NO: 2.

21. The method of claim 14, wherein the aberrant K4 polynucleotide comprises the sequence set forth in any one of SEQ ID NO; 1, 3 and 14.

22. The method of claim 14, wherein the presence of said aberrant KLK4 polynucleotide or said expression product is detected in a basal cell.

23. The method of claim 14, wherein the presence of said aberrant KLK4 polynucleotide or said expression product is detected in a basal cell of prostatic origin.

24. The method of claim 14, wherein the presence or said aberrant KLK4 polynucleotide or said expression product is detected in a stem cell of prostatic origin.

25. The method of claim 14, wherein the presence of said aberrant KLK4 polynucleotide or said expression product is detected in a stem cell of prostatic origin which is a precursor of, or differentiates into, an epithelial cell or a malignant cancer cell.

26. The method of claim 14, wherein the presence of said aberrant KLK4 polynucleotide or said expression product is detected in a precursor lesion to cancer.

27. The method of claim 14, wherein the presence of said aberrant KLK4 polynucleotide or said expression product is detected in a prostatic intra-epithelial neoplasia (PIN).

28. The method of claim 14, wherein the presence of said aberrant KLK4 polynucleotide or said expression product is detected in a bone metastasis.

29. The method of claim 14, wherein the presence of said aberrant KLK4 polynucleotide or said expression product is detected in a bone metastasis associated With a prostate cancer.

30. The use of an agent in the manufacture of a medicament for restoring a normal level and/or functional activity of a KLK4 expression product in a patient with a cancer or benign tumour associated with an organ or tissue selected from the group consisting of ovaries, endometrium and prostate and having an elevated level and/or functional activity of said expression product, wherein said agent is optionally formulated with a pharmaceutically acceptable carrier and modulates the expression of a gene or the level and/or functional activity of an expression product of said gene, wherein said gene is selected from KLK4 or a gene belonging to the same regulatory or biosynthetic pathway as KLK4, and is identifiable by a screening assay comprising:
containing a preparation comprising a polypeptide encoded by said gent, or a biologically active fragment of said polypeptide, or a variant or derivative of these, or a genetic sequence that modulates the expression of said gene, with said agent; and
detecting a reduction in tie level and/or functional activity or said polypeptide or biologically active fragment, or variant or derivative, or of a product expressed from said genetic sequence.

31. The use of claim 30, wherein said agent is an antigen-binding molecule that is immuno-interactive with a K4 polypeptide.

32. The use of claim 10, where said agent is an antisense oligonucleotide or ribozyme that binds to, or otherwise interacts specifically with, an aberrant KLK 4 transcript.

33. The use of an agent in the manufacture of a medicament for modulating the level and or functional activity of an aberrant K4 expression product in a patient which aberrant expression product is selected from the group consisting of an aberrant K4 polypeptide with impaired, altered or abrogated function relative to normal K4, wherein said aberrant K4 polypeptide comprises tie sequence set forth in any one of SEQ ID NO: 2, 4 and 15, or an aberrant K4 polynucleotide encoding said aberrant K4 polypeptide, wherein said agent is optionally formulated with a pharmaceutically acceptable carrier and is identifiable by a screening assay comprising:
contacting a preparation comprising an aberrant K4 polypeptide comprising the sequence set forth in any one of SEQ ID NO: 2, 4 and 15, or a biologically active fragment thereof; or a variant or derivative of these, or an alt K4 transcript that encodes said aberrant K4 polypeptide, with said agent, and
detecting a change in the level and/or functional activity of said polypeptide or biologically active fragment, or variant or derivative, or said aberrant KLK4 transcript.

34. The use of claim 33, wherein said agent is an antigen-binding molecule that is immuno-interactive with an aberrant K4 polypeptide.

35. The use of an agent in the manufacture of a medicament for treating and/or preventing a cancer or benign tumour associated with n organ or tissue selected from the group consisting of ovaries, endometrium and prostate, wherein sad agent modulates the expression of a gene or the level and/or functional activity of an expression product of said gene, wherein said gene is selected from KLK4 or a gene belonging to the same regulatory or biosynthetic pathway as KLK4, and is identifiable by a screening assay comprising:
contacting a preparation comprising a polypeptide encoded by said gone, or biologically active fragment of said polypeptide, or variant or derivative of these, or a genetic sequence that modulates the expression of said gene, with said agent; and
detecting a change in the level and/or functional activity of said polypeptide or biologically active fragment thereof; or variant or derivative, or of a product expressed from said genetic sequence.

36. A method for restoring a normal level of a KLK4 expression product in a patient with a cancer or benign tumour associated with an organ or tissue selected front the group consisting of ovaries, endometrium and prostate and having an elevated level and/or functional activity of said expression product, comprising administering to said patient all effective amount of an agent that reduces the level and or functional activity of said expression product, wherein said agent is optionally formulated with a pharmaceutically acceptable carrier and is identifiable by a screening assay comprising:
contacting a preparation comprising a polypeptide encoded by said gene, or a biologically active fragment of said polypeptide, or a variant or derivative of these, or a genetic sequence that modulates the expression of said gene, with said agent; and
detecting a reduction in the level and/or functional activity of said polypeptide or biologically active fragment, or variant or derivative, or of a product expressed from said genetic sequence.

37. A method for the treatment and/or prophylaxis of a cancer or benign tumour associated with an organ or tissue selected from the group consisting of ovaries, endometrium and prostate, comprising administering to a patent in need of such treatment an effective amount of an agent that modulates the level and or functional activity of a KLK4 expression product, wherein said agent is optionally formulated with a pharmaceutically acceptable carrier and is identifiable by a screening assay comprising:
contacting a preparation comprising a polypeptide encoded by said gene, or a biologically active fragment of said polypeptide, or a variant or derivative of these, or a genetic sequence that modulates the expression of said gene, with said agent; and
detecting a change in the level and/or functional activity of said polypeptide or biologically active fragment, or variant or derivative, or of a product expressed from said genetic sequence.

38. A method for the treatment and/or prophylaxis of a cancer or benign tumour associated with an organ or tissue selected from the group consisting of ovaries, endometrium and prostate, comprising administering to a patient in need of such treatment an effective amount of an agent that modulates the level and or functional activity of an aberrant KLK4 expression product selected from the group consisting of an aberrant K4 polypeptide with impaired, altered or abrogated function relative to normal K4, wherein said aberrant K4 polypeptide comprises the sequence set forth in any one of SEQ ID NO 2, 4 and 15, or an aberrant K4 polynucleotide encoding said aberrant K4 polypeptide, wherein said agent is optionally formulated with a pharmaceutically acceptable carrier and is identifiable by a screening away comprising:
contacting a preparation comprising an aberrant K4 polypeptide comprising the sequence set forth in any one of SEQ ID NO: 2, 4 and 15, or a biologically active fragment thereof, or a variant or derivative of these, or an aberrant KLK4 transcript that encodes said aberrant K4 polypeptide, with said agent; and
detecting a change in the level and/or functional activity of said polypeptide or biologically active fragment, or variant or derivative or said aberrant KLK4 transcript.

39. An isolated polynucleotide comprising a nucleotide sequence which corresponds or is complementary to at lest a portion of an aberrant KLK4 polynucleotide that correlates with the presence or risk of at least one condition selected from a cancer or a benign tumour, wherein said aberrant KLK4 polynucleotide is selected from the group consisting of:
(i) an aberrant KLK4 polynucleotide comprising all or part of the intron located between exon 3 and exon 4 of normal KLK4 as set forth in SEQ ID NO: 12;
(ii) an aberrant KLK4 polynucleotide comprising a deletion corresponding to all or part of exon 4 of normal KLK4 as set forth in SEQ ID NO: 12; and
(iii) an aberrant KLK4 polynucleotide comprising all or part of the intron located between exon 2 and axon 3 of normal 4 as sat forth in SEQ ID NO: 17.

40. The polynucleotide of claim 39, wherein said at least a portion of said aberrant KLK4 polynucleotide comprises at least 10 nucleotides.

41. The polynucleotide of claim 39, wherein said aberrant KLK4 polynucleotide of (i) comprises the nucleotide sequence set forth in SEQ ID NO: 7.

42. The polynucleotide of claim 39, wherein said aberrant KLK4 polynucleotide of (i) comprises a 3′ coding sequence comprising the sequence set forth in SEQ ID NO: 8.

43. The polynucleotide of claim 42, wherein said aberrant KLK4 polynucleotide comprises the sequence set forth in SEQ ID NO: 1.

44. The polynucleotide of claim 39, wherein said deletion of (ii) comprises all or part of the sequence set forth in SEQ D NO: 10.

45. The polynucleotide of claim 39, wherein said aberrant KLK4 polynucleotide of (ii) comprises all or part of a sequence corresponding to exon 3 of normal KLK4 spliced together with all or part of a sequence corresponding to exon 5 of normal KLK4.

46. The polynucleotide of claim 45, wherein said aberrant Kr C4 polynucleotide comprises the sequence set forth in SEQ ID NO: 18.

47. The polynucleotide of claim 45, wherein said aberrant KLK4 polynucleotide comprises the sequence set forth in SEQ ID NO: 3.

48. The polynuclotide of claim 39, wherein said aberrant KLK4 polynucleotide of (iii) comprises the intronic sequence set forth in SEQ D NO, 16.

49. The polynucleotide of claim 39, wherein said aberrant K 4 polynucleotide of (iii) further comprises a deletion corresponding to all or part of exon 4 of normal KLK4 as set forth in SEQ ID NO: 12.

50. The polynucleotide of claim 3, wherein said aberrant KLK4 polynucleotide of (iii) comprises all or part of a sequence corresponding to exon 3 of normal K4 spliced together with all or part of a sequence corresponding to exon 5 of normal KLK4.

51. The polynucleotide of claim 50, wherein said aberrant KLK4 polynucleotide comprises the sequence set forth in SEQ ID NO: 18.

52. The polynucleotide of claim 50, wherein said aberrant KLK4 polynucleotide preferably comprises the nucleotide sequence set forth in SEQ ID NO: 14.

53. The polynucleotide of claim 39, wherein said aberrant KLK4 polynucleotide is selected from the group consisting of:
(a) a polynucleotide comprising the entire sequence of nucleotides set forth in SEQ ID NO: 1;
(b) a polynucleotide fragment of (a), wherein said fragment comprises SEQ ID NO: 7 or fragment thereof;
(c) a polynucleotide comprising the entire sequence of nucleotides set forth in SEQ ID NO: 3;
(d) a polynucleotide fragment of (c), wherein said fragment comprises the codon spanning nucleotides 475 through 477 of SEQ ID NO: 3;
(e) a polynucleotide fragment of (c), wherein said fragment comprises all or part of a sequence corresponding to exon 3 of normal KLK4 spliced together with all or part of a sequence corresponding to exon 5 of normal KLK4;
(f) a polynucleotide comprising the entire sequence of nucleotides set forth in SEQ ID NO, 14;
(g) a polynucleotide fragment of (1), wherein said fragment comprises SEQ ID NO: 17, or pardon thereof;
(h) a polynucleotide fragment of (i), wherein said fragment comprises the codon sp n nucleotides 223 through 225 of SEQ ID NO: 14; and
(i) a polynucleotide fragment of (f), wherein said fragment comprises all or part of a sequence corresponding to exon 3 of normal KLK4 spliced together with all or part of a sequence corresponding to exon 5 of normal 14.

54. A probe for interrogating nucleic acid for the presence of an aberrant K4 polynucleotide that correlates with the presence or risk of a cancer or benign tumour associated with an organ or tissue selected from the group consisting of ovaries, endometrium and prostate, comprising a nucleotide sequence which corresponds or is complementary to a portion of the aberrant KLK4 polynucleotide of claim 39.

55. A vector comprising the polynucleotide of claim 39, or the probe of claim 54.

56. An expression vector c g the polynucleotide of claim 39, or the probe of claim 54, operably linked to a regulatory polynucleotide.

57. A host cell containing the vector of claim 55 or the expression vector of claim 56.

58. A cell line comprising a polynucleotide comprising a nucleotide sequence which corresponds or is complementary to at least a portion of the aberrant KLK4 polynucleotide of claim 39.

59. The cell line of claim 58, which is derived from a patient with a cancer or benign tumour associated with an organ or tissue selected from the group consisting of ovaries, endometrium and prostate.

60. An isolated polypeptide comprising an amino acid sequence which corresponds to at least a portion of an aberrant K4 polypeptide that correlates with the presence or risk of a cancer or benign tumour associated with an organ or tissue selected tom the group consisting of ovaries, endometrium and prostate, wherein the aberrant K4 polypeptide is selected from:
(a) an aberrant K4 polypeptide comprising an insertion relative to normal K4, which comprises the sequence set forth in SEQ ID NO: 9;
(b) an aberrant K4 polypeptide comprising comprises a truncation relative to normal K4, wherein said truncation is associated with a deletion of all or part of the amino acid sequence set forth in SEQ ID NO: 11; and
(c) an aberra=K4 polypeptide comprising comprises a truncation relative to normal K4, wherein said truncation is associated with a deletion of all or part of the amino acid sequence set fort in SEQ ID NO: 19.

61. The polypeptide of claim 60, wherein said aberrant K4 polypeptide of (a) comprises the sequence set forth in SEQ ID NO: 2.

62. The polypeptide of claim 60, wherein said aberrant K4 polypeptide of (b) comprises the sequence set forth in SEQ ID NO: 4.

63. The polypeptide of claim 60, wherein said aberrant K4 polypeptide of (c) comprises the sequence set forth in SEQ ID NO: 15.

64. An anti-binding molecule that is immuno-interactive specifically with a portion of the polypeptide of claim 60.

65. The use of the polynucleotide of claim 39 or the use of the probe of claim 54 or the use of the polypeptide of claim 60 or the use of the antigen-binding molecule of claim 64 for detecting an aberrant KLK4 polynucleotide, or an aberrant K4 polypeptide that correlates a cancer or benign tumour associated with an organ or tissue selected from the group consisting of ovaries, endometrium and prostate.
